SEQUENCE LISTING (104 sequences)

SEQ ID NO: 1 - DNA, 21 bp, Artificial Sequence
  Sepsis associated G(+) bacteria-specific probe
yttvttyagttttgagaggty

SEQ ID NO: 2 - DNA, 21 bp, Artificial Sequence
  Sepsis associated G(+) bacteria-specific probe
tgtattcagttttgaatgtty

SEQ ID NO: 3 - DNA, 22 bp, Artificial Sequence
  Sepsis associated G(-) bacteria-specific probe
ctcagttggttagagcgcwmcm

SEQ ID NO: 4 - DNA, 22 bp, Artificial Sequence
  Sepsis associated G(-) bacteria-specific probe
yrgkyctrtagctcagttggtt

SEQ ID NO: 5 - DNA, 22 bp, Artificial Sequence
  Sepsis associated G(-) bacteria-specific probe
tgagawrtttgctctttaamaa

SEQ ID NO: 6 - DNA, 22 bp, Artificial Sequence
  Sepsis associated G(-) bacteria-specific probe
attgtcttatgttctttaaaaa

SEQ ID NO: 7 - DNA, 21 bp, Artificial Sequence
  Sepsis associated bacteria genus-specific probe
acactgataatgtagaggtcg

SEQ ID NO: 8 - DNA, 20 bp, Artificial Sequence
  Sepsis associated bacteria genus-specific probe
gttcgaatccgttattctcc

SEQ ID NO: 9 - DNA, 17 bp, Artificial Sequence
  Sepsis associated bacteria genus-specific probe
gtagaggtcggcagttc

SEQ ID NO: 10 - DNA, 19 bp, Artificial Sequence
  Sepsis associated bacteria genus-specific probe
gagcgctacactgataatg

SEQ ID NO: 11 - DNA, 23 bp, Artificial Sequence
  Sepsis associated bacteria genus-specific probe
gagttttttaataagttcaattg

SEQ ID NO: 12 - DNA, 23 bp, Artificial Sequence
  Sepsis associated bacteria genus-specific probe
cgcgttgaatgagttttttaata

SEQ ID NO: 13 - DNA, 21 bp, Artificial Sequence
  Sepsis associated bacteria genus-specific probe
cgaatccccgtggggacgcca

SEQ ID NO: 14 - DNA, 21 bp, Artificial Sequence
  Sepsis associated bacteria genus-specific probe
aaagaacctgcctttgtagtg

SEQ ID NO: 15 - DNA, 22 bp, Artificial Sequence
  Sepsis associated bacteria genus-specific probe
tgaaaattgaaacgacacacag

SEQ ID NO: 16 - DNA, 17 bp, Artificial Sequence
  Sepsis associated bacteria genus-specific probe
aacctgcctttgtagtg

SEQ ID NO: 17 - DNA, 20 bp, Artificial Sequence
  Sepsis associated bacteria genus-specific probe
taatgtgtgttcgagtctct

SEQ ID NO: 18 - DNA, 20 bp, Artificial Sequence
  Sepsis associated bacteria genus-specific probe
gttctttgaaaactagataa

SEQ ID NO: 19 - DNA, 20 bp, Artificial Sequence
  Sepsis associated bacteria genus-specific probe
gaaagttagtaaagttagca

SEQ ID NO: 20 - DNA, 23 bp, Artificial Sequence
  Sepsis associated bacteria genus-specific probe
cacaagtaaccgagaatcatctg

SEQ ID NO: 21 - DNA, 21 bp, Artificial Sequence
  Sepsis associated bacteria genus-specific probe
cccacacgaattgcttgattc

SEQ ID NO: 22 - DNA, 20 bp, Artificial Sequence
  Sepsis associated bacteria genus-specific probe
tttcggcgaatgtcgtcttc

SEQ ID NO: 23 - DNA, 20 bp, Artificial Sequence
  Sepsis associated bacteria genus-specific probe
acagtataaccagattgctt

SEQ ID NO: 24 - DNA, 20 bp, Artificial Sequence
  Sepsis associated bacteria genus-specific probe
cagattgcttggggttatat

SEQ ID NO: 25 - DNA, 22 bp, Artificial Sequence
  Sepsis associated bacteria genus-specific probe
cacctccttacctaawgatatt

SEQ ID NO: 26 - DNA, 20 bp, Artificial Sequence
  Sepsis associated bacteria genus-specific probe
cctccttacctaawgatatt

SEQ ID NO: 27 - DNA, 21 bp, Artificial Sequence
  Sepsis associated bacteria genus-specific probe
gacatattgtattcagttttg

SEQ ID NO: 28 - DNA, 21 bp, Artificial Sequence
  Sepsis associated bacteria genus-specific probe
agattttaccaagcaaaaccg

SEQ ID NO: 29 - DNA, 20 bp, Artificial Sequence
  Sepsis associated bacteria genus-specific probe
cattgaaaattgaatawcka

SEQ ID NO: 30 - DNA, 20 bp, Artificial Sequence
  Sepsis associated bacteria genus-specific probe
attgaatawckatatcaaat

SEQ ID NO: 31 - DNA, 20 bp, Artificial Sequence
  Sepsis associated bacteria species-specific probe
gatattttatcttgtatgat

SEQ ID NO: 32 - DNA, 19 bp, Artificial Sequence
  Sepsis associated bacteria species-specific probe
tccgataccgcgacctaac

SEQ ID NO: 33 - DNA, 20 bp, Artificial Sequence
  Sepsis associated bacteria species-specific probe
ggcttgcgtgtaatcaaacg

SEQ ID NO: 34 - DNA, 20 bp, Artificial Sequence
  Sepsis associated bacteria species-specific probe
gaaaaggagatgaatctggc

SEQ ID NO: 35 - DNA, 20 bp, Artificial Sequence
  Sepsis associated bacteria species-specific probe
ctaacatcaggtagacaagg

SEQ ID NO: 36 - DNA, 20 bp, Artificial Sequence
  Sepsis associated bacteria species-specific probe
ctccgaacaaaaattgtcgt

SEQ ID NO: 37 - DNA, 18 bp, Artificial Sequence
  Sepsis associated bacteria species-specific probe
ggttaatacctgatactt

SEQ ID NO: 38 - DNA, 20 bp, Artificial Sequence
  Sepsis associated bacteria species-specific probe
tgtcgtaactgtcacaaacg

SEQ ID NO: 39 - DNA, 20 bp, Artificial Sequence
  Sepsis associated bacteria species-specific probe
tgacagccacttaaagactt

SEQ ID NO: 40 - DNA, 21 bp, Artificial Sequence
  Sepsis associated bacteria species-specific probe
ccagtatgagaataaaacgtt

SEQ ID NO: 41 - DNA, 19 bp, Artificial Sequence
  Sepsis associated bacteria species-specific probe
cgtctttactttgttcagt

SEQ ID NO: 42 - DNA, 19 bp, Artificial Sequence
  Sepsis associated bacteria species-specific probe
gcttatttattgattaacc

SEQ ID NO: 43 - DNA, 20 bp, Artificial Sequence
  Sepsis associated bacteria species-specific probe
agactacacaatttgttttt

SEQ ID NO: 44 - DNA, 20 bp, Artificial Sequence
  Sepsis associated bacteria species-specific probe
cttgatctaacttctatcgc

SEQ ID NO: 45 - DNA, 20 bp, Artificial Sequence
  Sepsis associated bacteria species-specific probe
catgggaattagtaagaccc

SEQ ID NO: 46 - DNA, 19 bp, Artificial Sequence
  Sepsis associated bacteria species-specific probe
actaaggaacatgatcgct

SEQ ID NO: 47 - DNA, 20 bp, Artificial Sequence
  Sepsis associated bacteria species-specific probe
ggatacagaaacaattttaa

SEQ ID NO: 48 - DNA, 18 bp, Artificial Sequence
  Sepsis associated bacteria species-specific probe
tgagactgtacgtcccct

SEQ ID NO: 49 - DNA, 20 bp, Artificial Sequence
  Sepsis associated bacteria species-specific probe
agtgaaagtcacctgccgtc

SEQ ID NO: 50 - DNA, 20 bp, Artificial Sequence
  Sepsis associated bacteria species-specific probe
aagtagagaagcaaggggtc

SEQ ID NO: 51 - DNA, 22 bp, Artificial Sequence
  Sepsis associated bacteria species-specific probe
agtgaaagacgcgtgccgatat

SEQ ID NO: 52 - DNA, 22 bp, Artificial Sequence
  Sepsis associated bacteria species-specific probe
gataccttcccgcgcagtgtcc

SEQ ID NO: 53 - DNA, 20 bp, Artificial Sequence
  Sepsis associated bacteria species-specific probe
gtgtctccatacagtatctc

SEQ ID NO: 54 - DNA, 20 bp, Artificial Sequence
  Sepsis associated bacteria species-specific probe
tgagcaggacggctgccaag

SEQ ID NO: 55 - DNA, 20 bp, Artificial Sequence
  Sepsis associated bacteria species-specific probe
ccaagtcgtgacacatttgt

SEQ ID NO: 56 - DNA, 21 bp, Artificial Sequence
  Sepsis associated bacteria species-specific probe
aaagaagcgtwctttgmagtg

SEQ ID NO: 57 - DNA, 21 bp, Artificial Sequence
  Sepsis associated bacteria species-specific probe
atctcaaaactcatcttcggg

SEQ ID NO: 58 - DNA, 21 bp, Artificial Sequence
  Sepsis associated bacteria species-specific probe
aaaactcatcttcgggtgatg

SEQ ID NO: 59 - DNA, 19 bp, Artificial Sequence
  Sepsis associated bacteria species-specific probe
ctttattagattgtcttac

SEQ ID NO: 60 - DNA, 21 bp, Artificial Sequence
  Sepsis associated bacteria species-specific probe
gagagaaagtctgagtaggca

SEQ ID NO: 61 - DNA, 20 bp, Artificial Sequence
  Sepsis associated bacteria species-specific probe
aatcaagtgtttagttgaat

SEQ ID NO: 62 - DNA, 21 bp, Artificial Sequence
  Sepsis associated bacteria species-specific probe
gacaagattaaaaacgaagcg

SEQ ID NO: 63 - DNA, 20 bp, Artificial Sequence
  Sepsis associated bacteria species-specific probe
taaagacatcacaagtactc

SEQ ID NO: 64 - DNA, 19 bp, Artificial Sequence
  Sepsis associated bacteria species-specific probe
gattgtttgattgttttag

SEQ ID NO: 65 - DNA, 21 bp, Artificial Sequence
  Sepsis associated bacteria species-specific probe
aagtagaaagtctgagtaatc

SEQ ID NO: 66 - DNA, 20 bp, Artificial Sequence
  Sepsis associated bacteria species-specific probe
agctaagtgtttagtctaaa

SEQ ID NO: 67 - DNA, 18 bp, Artificial Sequence
  Sepsis associated bacteria species-specific probe
gacgcgtgccgatgtatc

SEQ ID NO: 68 - DNA, 20 bp, Artificial Sequence
  Sepsis associated bacteria species-specific probe
ttgagacttcagtgtcccct

SEQ ID NO: 69 - DNA, 20 bp, Artificial Sequence
  Sepsis associated bacteria species-specific probe
acgcgtgccgawstatctca

SEQ ID NO: 70 - DNA, 20 bp, Artificial Sequence
  Sepsis associated bacteria species-specific probe
gctgcgaagtcgcgacacct

SEQ ID NO: 71 - DNA, 20 bp, Artificial Sequence
  Sepsis associated bacteria species-specific probe
gcgacacgacgatgttttac

SEQ ID NO: 72 - DNA, 19 bp, Artificial Sequence
  Sepsis associated bacteria species-specific probe
gcacaaccaaccgatacct

SEQ ID NO: 73 - DNA, 24 bp, Artificial Sequence
  Sepsis associated bacteria species-specific probe
catagataatttattatttatgac

SEQ ID NO: 74 - DNA, 20 bp, Artificial Sequence
  Sepsis associated bacteria species-specific probe
cactctcttagatgtcagat

SEQ ID NO: 75 - DNA, 20 bp, Artificial Sequence
  Sepsis associated bacteria species-specific probe
ctgtataacctatttaaggg

SEQ ID NO: 76 - DNA, 20 bp, Artificial Sequence
  Sepsis associated bacteria species-specific probe
gaaactttccgctttggaag

SEQ ID NO: 77 - DNA, 19 bp, Artificial Sequence
  Sepsis associated bacteria species-specific probe
cttacgcaatcgcgtaaat

SEQ ID NO: 78 - DNA, 21 bp, Artificial Sequence
  Sepsis associated bacteria species-specific probe
gaaacttgcgtttttcattct

SEQ ID NO: 79 - DNA, 21 bp, Artificial Sequence
  Sepsis associated bacteria species-specific probe
cgcaaggctacatgctctgga

SEQ ID NO: 80 - DNA, 21 bp, Artificial Sequence
  Sepsis associated bacteria species-specific probe
agaaaacaaaatattatttcc

SEQ ID NO: 81 - DNA, 20 bp, Artificial Sequence
  Sepsis associated bacteria species-specific probe
ctgataccaacgattagaaa

SEQ ID NO: 82 - DNA, 18 bp, Artificial Sequence
  Sepsis associated bacteria species-specific probe
gcttgattcactggttag

SEQ ID NO: 83 - DNA, 19 bp, Artificial Sequence
  Sepsis associated bacteria species-specific probe
ccatctaaaacaatcgtcg

SEQ ID NO: 84 - DNA, 19 bp, Artificial Sequence
  Sepsis associated bacteria species-specific probe
acccgagagtaacgattgg

SEQ ID NO: 85 - DNA, 19 bp, Artificial Sequence
  Sepsis associated bacteria species-specific probe
ccattaactctagtcgccg

SEQ ID NO: 86 - DNA, 19 bp, Artificial Sequence
  Sepsis associated bacteria species-specific probe
cacgttatagacagtaacc

SEQ ID NO: 87 - DNA, 20 bp, Artificial Sequence
  Sepsis associated bacteria species-specific probe
aagatattagttcgagtggc

SEQ ID NO: 88 - DNA, 21 bp, Artificial Sequence
  Sepsis associated bacteria species-specific probe
tagttcgagtggcgtgctcac

SEQ ID NO: 89 - DNA, 21 bp, Artificial Sequence
  Sepsis associated bacteria species-specific probe
gatattgattgcgtgaagtgc

SEQ ID NO: 90 - DNA, 21 bp, Artificial Sequence
  Sepsis associated bacteria species-specific probe
gatattgattcgagtgaagtg

SEQ ID NO: 91 - DNA, 22 bp, Artificial Sequence
  Sepsis associated bacteria species-specific probe
tcacaagattaataacgcgttt

SEQ ID NO: 92 - DNA, 20 bp, Artificial Sequence
  Sepsis associated bacteria species-specific probe
ttgaattcvtaaataatcgc

SEQ ID NO: 93 - DNA, 20 bp, Artificial Sequence
  Sepsis associated bacteria species-specific probe
ctagttttagctatttattt

SEQ ID NO: 94 - DNA, 19 bp, Artificial Sequence
  Sepsis associated bacteria species-specific probe
cttacgaagatgcaggaat

SEQ ID NO: 95 - DNA, 20 bp, Artificial Sequence
  Sepsis associated bacteria species-specific probe
caaccgacttgttcgtgttg

SEQ ID NO: 96 - DNA, 21 bp, Artificial Sequence
  Sepsis associated bacteria species-specific probe
aggaaacctgccatttgcgtc

SEQ ID NO: 97 - DNA, 21 bp, Artificial Sequence
  Sepsis associated bacteria species-specific probe
agttttctagttttaaagaaa

SEQ ID NO: 98 - DNA, 20 bp, Artificial Sequence
  Sepsis associated bacteria species-specific probe
aaggaactgcgcattggtct

SEQ ID NO: 99 - DNA, 21 bp, Artificial Sequence
  Sepsis associated bacteria species-specific probe
atcaccaagtaatgcacattg

SEQ ID NO: 100 - DNA, 22 bp, Artificial Sequence
  Sepsis associated bacteria species-specific probe
acacgtttatcgtcttatttag

SEQ ID NO: 101 - DNA, 20 bp, Artificial Sequence
  Sepsis associated bacteria species-specific probe
cgatctagaaatagattgta

SEQ ID NO: 102 - DNA, 22 bp, Artificial Sequence
  Sepsis associated bacteria species-specific probe
acggaagcacgtttgggtattg

SEQ ID NO: 103 - DNA, 20 bp, Artificial Sequence
  Sepsis associated bacteria species-specific probe
attcaaagattgtccattga

SEQ ID NO: 104 - DNA, 21 bp, Artificial Sequence
  Sepsis associated bacteria species-specific probe
gtttaaggtcaacagaaccaa

(19) United States
(12) Patent Application Publication
(10) Pub. No.: US 2009/0286691 A1
(43) Pub. Date: Nov. 19, 2009

(54) Oligonucleotide for Detection of Bacteria Associated with Sepsis and Microarrays and Method for Detection of the Bacteria Using the Oligonucleotide

(75) Inventors: Cheol-Min Kim, Busan (KR); Hee-Kyung Park, Seoul (KR); Hyun-Jung Jang, Busan (KR); Eun-Sil Song, Busan (KR)

Correspondence Address: THE RAFFERTY PATENT LAW FIRM, 1952 Gallows Road, Suite 200, Vienna, VA, 22182-3823, US

(73) Assignee: GENEIN CO., LTD., Busan (KR)

(21) Appl. No.: 12/161,691

(22) PCT Filed: Jan. 20, 2006
(86) PCT No.: PCT/KR2006/000237

§ 371 Date: Jul. 22, 2008

(51) Int. Cl. C40B 30/04; C07H 21/04; C12Q 1/68; C40B 40/06; C40B 40/08

(52) U.S. Cl. 506/9; 536/24.33; 536/24.32; 536/23.1; 435/6; 506/16; 506/17

(57) ABSTRACT

The present invention relates to oligonucleotides for detection of sepsis-causing bacteria and a detection method using the oligonucleotides, more particularly to a microarry comprising at least one of gram positive bacteria-specific and gram negative bacteria-specific oligonucleotides, sepsis-causing bacteria's genus-specific and species-specific oligonucleotides designed from the ITS target region which is hypervariable base sequence of sepsis-causing bacteria as probes, and a detection method and a diagnosis kit by using the same.

According to the present invention, the present invention can provide an antibiotics therapy for accurately removing infectious agent related to sepsis by detecting existence of sepsis-causing bacteria and identifying gram positive- and gram negative-bacteria and genus and species of the bacteria, at once. And, the present invention can prevent a patient from abuse and misuse of antibiotics and decrease time of hospital treatment and medical cost of the patient. Further, the present invention has advantage of preventing complications and reducing mortality rate.

### TECHNICAL FIELD

The present invention relates to oligonucleotides useful for detection and differential diagnosis of sepsis-causing bacteria and a detection method using the same, more particularly to a microarry comprising at least one of gram positive bacteria-specific and gram negative bacteria-specific oligonucleotides, sepsis-causing bacteria's genus-specific and species-specific oligonucleotides designed from the ITS target region which is hypervariable sequence of sepsis-causing bacteria as probes, and a detection method and a diagnosis kit using the same.

### BACKGROUND ART

Sepsis means a disease in which systemic inflammatory response syndrome (SIRD) is occurred by several microorganisms. Sepsis can be occurred by enterence of microorganisms living together in stomach tube or tissue closed by skin, and partially infection of microorganisms in urogenital organ, bile, lung or stomach tube can induce blood infection. Microorganisms also can be directly infiltrated into blood through an intravenous injection. In other words, a host human reaction appears variously by infection of such microorganisms, it is noticed as inflammation response such as high fever, slight fever, rigor, tachycardia and tachypnea. The sepsis is a very fatal disease that progresses as severe sepsis, septic shock or MODS (multiple organ dysfunction syndrome) which brings about dysfunction of lung, kidney, liver and circulatory organ as a complication and makes a patient reach to the death when it can not diagnose the reason accurately in an early period. Therefore, it is very important that causing microorganisms are accurately separated from blood or infection region to diagnose sepsis accurately.

Sepsis can appear in everyone infected by the microorganism, especially sepsis appears as high frequency in inpatients such as a newborn baby, an old person, the people having lack of immunity, the people having a burn, the people having a wound by a traffic accident, an alcohol addict, drug addict and the people who takes a treatment using catheter in hospital.

Sepsis is a cause that at least the 18 million persons progress to a severe sepsis every year, as sepsis is a typical incurable disease with 20˜50% of mortality rate, and sepsis is a main reason of 14 hundred persons' death daily, it is reported that the frequency is 5-10 times than rectal or beast cancer. More than 215 thousands persons in USA and more than 135 thousands persons in Europe have died by diseases associated with sepsis every year. Sepsis is in 10 kinds of death reason containing cancer in USA. Such seriousness is caused by aging society, life extension of a chronic patient and the problem of AIDS and so on, it is predicted to be increased continuously in Korea. Therefore, there have been needs to a rapid and accurate detection method which can detect sepsis-causing bacteria in an early stage and can protect from the bacteria ((Ann Internal Med, 115 (6):457-469 (1991), International Sepsis Forum: second edition (2003), 11 (2):32-34 (2004), Crit Care Med, 29 (7):S109-S116 (2001), Crit Care Med, 29 (7):1303-1310 (2001), N Engl J Med, 348:1546-1554 (2003). Sepsis is appeared by several microorganisms; sepsis is also occurred by fungi and virus, but most of causing microorganisms are bacteria. In 30˜60% of sepsis patient and 60˜80% of septic shock patient, their blood have two third of gram-negative bacteria and 10˜20% of gram-positive bacteria when culturing the blood. Recently, gram-positive bacteria are on the increase compared to gram-negative bacteria. According to difference of cell wall of gram-negative and gram-positive bacteria, immune activity is different, and then according to this, a prescription of antibiotics is different. Therefore, at first it is important to distinguish between gram-negative and gram-positive bacteria. Furthermore, it is the most important thing to identify exactly infected species in an early treatment of sepsis ((Crit Care Med, 27 (8):1608-1616 (1999).

Until now, it has been widely known to the method of culturing harvest from blood or infection region in the method for diagnosis of sepsis. However, this is easily to bring about remarkably low positive and pseudonegative results. Recently, detection is possible within 24˜48 hours by an automatic instrument for culturing, but it is high price and the media to request differs for each bacteria. Specially, the bacteria which has an experience to be injected by antibiotics, a growth rate is so slow and cultured slowly, and is particular about it's culturing condition, is hart to be detected (Clin Microbiol Rev, 10 (3):444-465 (1997)).

Now, DNA chip is in the spotlight as very useful technology, which can analyze many genes at once as they can attach a little genetic material on solid scaffold by using principle of pobe hybridization. This identification method using molecular biological detecting technology is a technically very progressive method which can rapidly, sensitively and exactly detect at once in a cultured and clinical sample from bacteria with very slow proliferation rate and hardly culturing condition.

As a commercially know method, BMS in Korea provides the method which can rapidly and conveniently detect pathogenic bacteria than the method which requires a lot of times and high level technology. But this method is a limit to detect various bacteria using 16S rDNA gene of very complemental sequence as target region to distinguish species.

According to leading technology detecting pathogenic bacteria and applied for a patent in Korea, the application NO. 10-2002-0016679 can detect pathogenic bacteria by using multiplex PCR. However, this can detect bacteria species less than bacteria species of the present invention, a main goal is the detection of pathogen which raises food poisoning and this is a drawback that has an effect on result of detection or has a contamination by a large number of primers in the method.

The Kor. Patent No. 10-2003-0005341, as other patent in Korea, shows DNA chip technology detecting bacteria associate with bacterial disease, this can detect 12 species of bacteria but there is no identical species to the present invention associated with sepsis. The Kor. Patent No. 10-2005-7018087, as another patent, relates to nucleic acid probes for detecting pathogenic bacteria more than 44 species using DNA chip technology such as the present invention. But there is no identical species to ITS target region of the present invention associated with sepsis. There is using not ITS target region but 23 rDNA gene of base sequence of little diversity in some species able to detect identically to the present invention. There is a limit to accuracy for detection, There is considerably different from detecting at once bacteria-specific, gram positive-specific, gram negative-specific, genus-specific and species-specific detection of the present invention because this is able to detect only species of bacteria.

There is a limit to detection base on 16S rDNA (J Microbiol Methods, 55:541-555 (2003), Pediatrics, 95:165-169 (1995), J Medi Microbiol, 52:685-691 (2003), J Clin Microbiol, 32:335-351 (1994), Microbiol, 148:257-266 (2002), Pediatr Res 58:143-148 (2005)) of very complementary base sequence of bacteria in target region to detect infectious bacteria in several reference. Recently, there is methods for detecting bacteria based on 23S rDNA (J Clin Microbiol, 38:781-788 (2000), J Microbiol Methods, 53:245-252 (2003), J Clin Microbiol, 42:1048-1057 (2004)) unexplained base sequence yet, but there is a limit to methods for detecting only some other bacteria or some species among one bacteria genus by this technology.

### DETAILED DESCRIPTION OF THE INVENTION

### Technical Goal of the Invention

To solve the above problem, the present invention provides oligonucleotides for detecting gram positive and gram negative sepsis-causing bacteria, originated from ITS having hypervariable region which is base sequences of many variation.

In addition, another object of the present invention is to provide a microarray comprising oligonucleotides as probes for detection of sepsis-causing bacteria.

In addition, another object of the present invention is to provide a method for identification and detection of sepsis-causing bacteria by using the microarray.

In addition, another object of the present invention is to provide a kit for identification and detection of sepsis-causing bacteria by using the microarray.

### Disclosure of the Invention

In order to achieve the object of the present invention, the present invention provides an oligonucleotide for gram positive-specific detection of sepsis-causing bacteria, comprising any one of one base sequence selected from SEQ ID Nos. 1 to 2 or its complementary sequence.

In order to achieve another object of the present invention, the present invention provides an oligonucleotide for gram negative-specific detection of sepsis-causing bacteria, comprising any one of one base sequence selected from SEQ ID Nos. 3 to 6 or its complementary sequence.

In order to achieve another object of the present invention, the present invention provides an oligonucleotide for genus-specific detection of sepsis-causing bacteria, comprising any one of one base sequence selected from SEQ ID Nos. 7 to 30 or its complementary sequence.

In order to achieve another object of the present invention, the present invention provides an oligonucleotide for species-specific detection of sepsis-causing bacteria, comprising any one of one base sequence selected from SEQ ID Nos. 31 to 104 or its complementary sequence.

The oligonucleotides of the present invention are designed based on multiple sequence alignment of ITS (internal transcribed spacer) hypervariable sequences of bacteria. The oligonucleotides have specificity to the gram positive and gram negative bacteria, and can be used as primers for PCR amplification or probes for hybridization in order to specifically detect genus and species of the bacteria.

In order to achieve another object of the present invention, the present invention provides a gram positive-specific and gram negative-specific probe set and a genus-specific and species-specific probe set for detection of sepsis-causing bacteria, comprising more than one oligonucleotide selected from the above oligonucleotides.

In order to achieve another object of the present invention, the present invention provides a kit for diagnosing gram positive-specific and gram negative-specific sepsis-causing bacteria and genus-specific and species-specific sepsis-causing bacteria, comprising more than one oligonucleotide selected from the above oligonucleotides.

In the kit of the present invention, the oligonucleotides may be labeled with radioactive or non-radioactive labeling agent, the latter comprises conventional biotin, Dig (digoxigenin), FRET (fluorescence resonance energy transfer) and fluorescent dye (Cy5 or Cy3). And, the oligonucleotides can be used as primers or probes and the kit can comprise another primers for PCR amplification of the target DNA.

In order to achieve another object of the present invention, the present invention provides a microarray comprising more than one oligonucleotide selected from oligonucleotides for gram positive-specific and gram negative-specific detection and genus-specific and species-specific detection of the sepsis-causing bacteria, as the probes attached on a support.

In the microarray of the present invention, the probes may be any materials having base sequence of the above oligonucleotides, preferably any one selected from a group consisting of DNA (Deoxyribose Nucleic Acid), RNA (Ribosse Nucleic Acid), and nucleic acid analogues selected from PNA (Peptide Nucleic Acid), LNA (Locked Nucleic Acid) and HNA (Hexitol Nucleic Acid). The nucleic acid analogues is stable to enzymes such as nuclease, has structurally specific interaction with base sequence, and has advantage of stability in heat.

In the microarray of the present invention, the probes can be manufactured for sense or antisense of the oligonucleotides. Therefore, the oligonucleotides have base sequence of the SEQ ID Nos. or its complementary sequence.

In the microarray of the present invention, the probes can further comprise bacteria specific nucleotides which is commonly present in bacteria and well-known in the art. For example, the probes can further comprise a bacteria specific oligonucleotide such as SEQ ID No. 46 of Kor. Patent No. 2004-68313, which is commonly present in 23S rDNA base sequence of the bacteria.

In the microarray of the present invention, the support may be made of any one selected from a group consisting of slide glass, plastic, membrane, semiconductive chip, silicon, gel, nano material, seramic, metal material and optical fiber or those mixture. The microarray of the present invention can be manufactured using conventional method such as a pin microarray (Microarray printing technology, Don Rose, Ph.D., Cartesian Technologies, Inc., Anal Biochem, 320 (2):281-91 (2003)), a ink jet (Nat Biotech, 18; 438-441 (2000), Bioconjug Chem, 13 (1); 97-103 (2002)), photolithography (Cur Opinion Chem Biol, 2; 404-410 (1998), Nature genetics supplement, 21:20-24 (1999)) or a electric array method (Ann Biomed Eng. 20 (4):423-37 (1992), Psychiatric Genetics, 12; 181-192 (2002)).

The microarray of the present invention, being provided in the form of diagnosis kit, may further comprise an extraction agent for isolating target DNA, a PCR kit containing primers for amplifying target gene, a hybridization reaction buffer, a washing solution for the unhybridized DNA, a cover slip, dyes, a washing solution for unbound dyes and a description sheet for the microarray, except for the microarray of the present invention.

In order to achieve another object of the present invention, the present invention provides a method for detection, comprising the following steps:
a) purifying nucleic acids from a sample;
b) amplifying target DNA among the purified nucleic acid;
c) hybridizing the amplified target DNA with probes of the microarray according to the present invention; and
d) detecting signals generated from the formed hybrid.

In the detection method of the present invention, the step b) for amplifying target DNA can be performed using Hot-start PCR, Nested PCR, Multiplex PCR, RT-PCR (reverse transcripase PCR), DOP (degenerate oligonucleotide primer) PCR, Quantitive RT-PCR, In-Situ PCR, Micro PCR, modified PCR such as Lab-on a chip PCR and isothermal amplification method such as RCA (rolling circle amplification) as well as general PCR reaction.

Also, the detection method of the present invention, with or without the step b) for amplifying target DNA, can be performed using probe amplification or signal amplification reaction such as tyramide signal amplification (Nucleic Acids Res. 30; e4 (2002)), nanoparticle probe, Raman-active dye (Science, 297; 1536-1540 (2002)) and branched DNA.

In the method of the present invention, the sample may be bloods, body fluids, tissues, sputum, excreta, urine or discharge. The purifying step a) can be performed using conventional DNA or RNA purification method or kit. The amplifying step b) can be performed using conventional PCR method. The detection of the PCR product can be performed using conventional electrophoresis with agarose gel. And, the signal detecting step d) can be performed using conventional fluorescence scanner after binding with conventional dyes such as Cy5 or Cy3.

According to the present invention, there can be provided a method for simultaneously genotying and detecting more than one sepsis-causing bacteria species selected from a group consisting of the following members:
a) gram positive bacteria (SEQ ID Nos. 1 to 2) and gram negative bacteria (SEQ ID Nos. 3 to 6);
b) Bacteroides genus (SEQ ID Nos. 7 to 10) and Bacteriodes species (SEQ ID Nos. 31 to 40);
c) Enterococcus genus (SEQ ID Nos. 11 to 12) and Enterococcus species (SEQ ID Nos. 41 to 47);
d) Enterobacter genus (SEQ ID Nos. 17) and Enterobacter species (SEQ ID Nos. 48 to 55);
e) Escherichia coli species (SEQ ID Nos. 56 to 58);
f) Haemophilus genus (SEQ ID Nos. 13) and Haemophilus species (SEQ ID Nos. 59 to 66);
g) Klebsiella genus (SEQ ID Nos. 14 to 17) and Klebsiella species (SEQ ID Nos. 67 to 72);
h) Listeria genus (SEQ ID Nos. 18 to 20) and Listeria species (SEQ ID Nos. 73 to 81);
i) Pseudomonas genus (SEQ ID Nos. 21 to 24) and Pseudomonas species (SEQ ID Nos. 82 to 86);
j) Serratia genus (SEQ ID Nos. 25 to 26) and Serratia species (SEQ ID Nos. 87 to 90);
k) Staphylococcus genus (SEQ ID Nos. 27 to 28) and Staphylococcus species (SEQ ID Nos. 91 to 95); and
l) Streptococcus genus (SEQ ID Nos. 29 to 30) and Streptococcus species (SEQ ID Nos. 96 to 104).

Therefore, the present invention provides a method for gram positive-specific and gram negative-specific detection of bateria associated with sepsis and a method for detecting one or more bacteria species simultaneously.

And, In order to achieve another object of the present invention, the present invention provides a method for detection using SBE (Single base extension), Sequencing, RFLP (Restriction fragment length polymorphism), or REA (Restriction endonuclease analysis) based on difference of one base sequence by using the oligonucleotides which is designed for gram positive-specific and gram negative-specific detection of sepsis-causing bacteria and for genus-specific and species-specific detection of sepsis-causing bacteria.

Hereafter, the present invention will be described in more detail.

The method for detecting existence of sepsis-causing bacteria and identifying gram positive-specific and gram negative bacteria and genus-specific and species-specific bacteria using the microarray of the present invention comprises the following steps:
a) if necessary, purifying nucleic acids from a cultured or clinical sample;
b) if necessary, amplifying the target sequence of bacteria or its part using more than one proper primers;
c) hybridizing the amplified target DNA with probes having a sense or antisense or complementary sequence of genus-specific and species-specific oligonucleotides and gram positive-specific and gram negative-specific oligonucleotides of sepsis-causing bacteria, disclosed in Tables 1 and 4;
d) detecting signals generated from the formed hybrid; and
e) predicting the existence of the sepsis-causing bacteria in the sample.

According to an example of the microarray of the present invention, in the c) step, the probes have a diversity of probe composition and more than one probes. In a particular embodiment, the probes are optimized to simultaneously hybrid with its target region under the same hybrid and washing condition which can detect gram positive and gram negative bacteria and genus and species of the sepsis-causing bacteria at once.

To achieve the object of the present invention, the present invention provides a microarray comprising probes set for detecting gram positive-specific and gram negative-specific bacteria and genus and species of the sepsis-causing bacteria attached on the support, which can simultaneously detect gram positive and gram negative bacteria and identify genus and species of the bacteria as quickly and exactly as passible from only one experiment of a sample.

Moreover, it is possible to detect a bacteria species which is not comprised in the microarray within at least level of genus. Furthermore, it is possible to detect existence of a bacteria species which is not pertained to the genus in the microarray.

### BRIEF DESCRIPTION OF THE DRAWINGS

FIG. 1 shows a schematic flow chart of the prevent invention.

FIG. 2 shows location of tartet region, primers and probes used for amplifying sepsis-causing bacteria from biological sample.

FIG. 3 show a microarray comprising a probe set consisting of bacteria universal probes and genus-specific and species-specific probes for detecting bacteria associated with sepsis, attached on a support.

FIG. 4 shows results of hybridization reaction of bacterial common probes and genus-specific and species-specific probes for Streptococcus bovis among sepsis-causing bacteria.

FIG. 5 shows results of hybridization reaction of bacterial common probes and genus-specific and species-specific probes for Staphylococcus saprophyticus among sepsis-causing bacteria.

FIG. 6 shows results of hybridization reaction of bacterial common probes and genus-specific and species-specific probes for Enterococcus faecium among sepsis-causing bacteria.

FIG. 7 shows results of hybridization reaction of bacterial common probes and genus-specific and species-specific probes for Listeria monocytogenes among sepsis-causing bacteria.

FIG. 8 shows results of hybridization reaction of bacterial common probes and genus-specific and species-specific probes for Escherichia coli among sepsis-causing bacteria.

FIG. 9 shows results of hybridization reaction of bacterial common probes and genus-specific and species-specific probes for Enterobacter aerogenes among sepsis-causing bacteria.

FIG. 10 shows results of hybridization reaction of bacterial common probes and genus-specific and species-specific probes for Klebsiella pneumoniae among sepsis-causing bacteria.

FIG. 11 shows results of hybridization reaction of bacterial common probes and genus-specific and species-specific probes for Serratia marcescens among sepsis-causing bacteria.

FIG. 12 shows results of hybridization reaction of bacterial common probes and genus-specific and species-specific probes for Pseudomonas aeruginosa among sepsis-causing bacteria.

FIG. 13 shows results of hybridization reaction of bacterial common probes and genus-specific and species-specific probes for Bacteroides thetaiotaomicron among sepsis-causing bacteria.

FIG. 14 shows results of hybridization reaction of bacterial common probes and genus-specific and species-specific probes for Streptococcus intermedius among sepsis-causing bacteria.

FIG. 15 shows results of hybridization reaction of bacterial common probes for Salmonella bongori unrelated with sepsis.

FIG. 16 shows results of hybridization reaction of bacterial common probes for Acinetobacter baumannii unrelated with sepsis.

FIG. 17 shows a microarray comprising a probe set consisting of bacteria universal probes, gram-positive and gram-negative probes and genus-specific and species-specific probes attached on a support for more specific detection of the sepsis-causing bacteria.

FIG. 18 shows results of hybridization reaction of bacterial common probes, gram positive-specific probes and genus-specific and species-specific probes for Streptococcus pneumoniae pertaining to gram-positive sepsis-causing bacteria.

FIG. 19 shows results of hybridization reaction of bacterial common probes and gram positive-specific probes for Bacillus cereus of gram-positive bacteria unrelated with sepsis.

FIG. 20 shows results of hybridization reaction of bacterial common probes, gram negative-specific probes and genus-specific and species-specific probes for Serratia marcescens pertaining to gram-negative sepsis-causing bacteria.

FIG. 21 shows results of hybridization reaction of bacterial common probes, gram negative-specific probes and genus-specific and species-specific probes for Pseudomonas stuzen pertaining to gram-negative sepsis-causing bacteria.

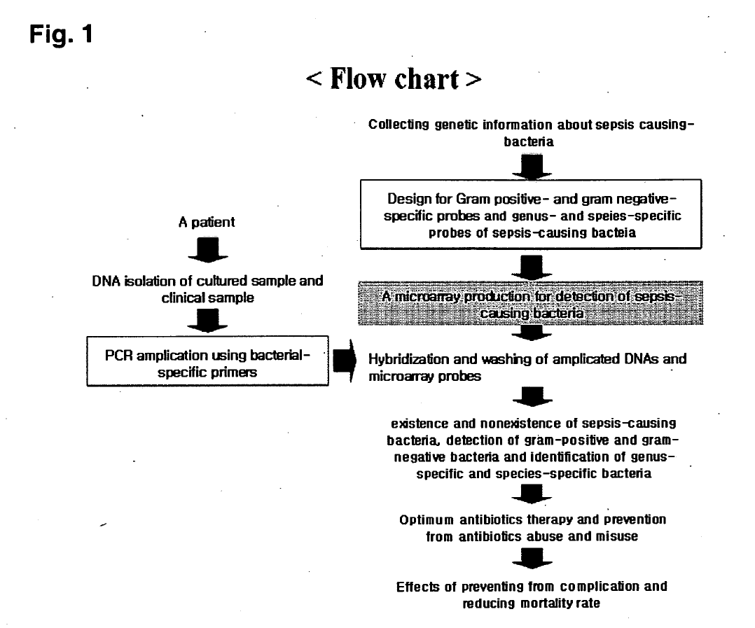

FIG. 1 shows a schematic flow chart of the prevent invention. In FIG. 1, the method according to the present invention comprises designing specific probes for detecting gram positive and gram negative sepsis-causing bacteria, extracting DNA from cultured and clinical sample, amplifying nucleic acid by PCR, and detecting accurately and simultaneously gram-positive and gram-negative bacteria and genus and species of bacteria with existence of sepsis-causing bacteria by using the microarray.

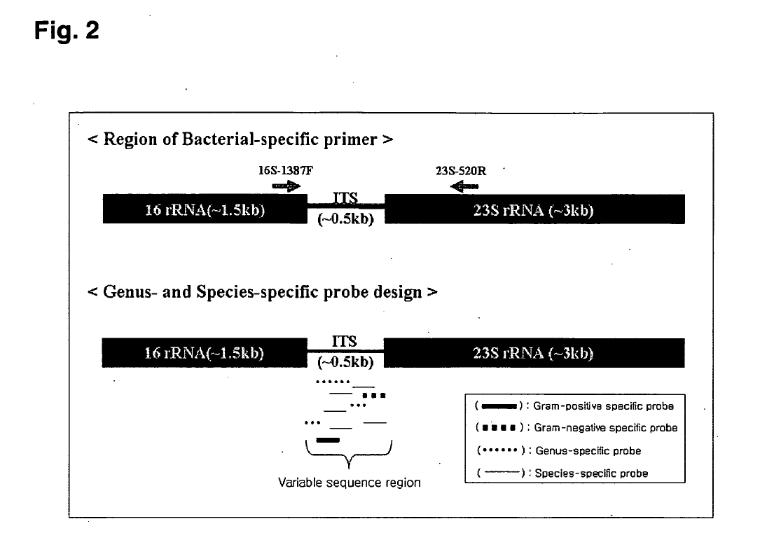

FIG. 2 shows location of the target region for amplifying bacteria in biological sample, primers for amplifying and probes. FIG. 2 shows, in gene structure of bacteria, ITS of base sequence hypervariable region, location of primers belong to bacteria which can amplify ITS, specific probes designed by present inventor for gram positive and gram negative bacteria and specific location of probes for genus and species.

New oligonucleotides for gram positive-specific probes of sepsis-causing bacteria developed in the present invention are as shown in Table 1.

TABLE 1

New probes for gram positive-specific detection
of sepsis-causing bacteria

| Target | Gram positive | | | Seq. ID |
|---|---|---|---|---|
| region | target Genus name | Probe | Base Sequence (5′ → 3′) | No. |
| ITS | Enterococcus | GP-01 | YTTVTTYAGTTTTGAGAGGTY | 1 |
| | Listeria | | | |
| | Staphylococcus | GP-02 | TGTATTCAGTTTTGAATGTTY | 2 |
| | Streptococcus | | | |

 Mixed Base∘| Code Name
V: G + A + C, Y: C + T

New oligonucleotides for gram negative-specific probes of sepsis-causing bacteria developed in the present invention are as shown in Table 2.

TABLE 2

New probes for gram negative-specific detection
of sepsis-causing bacteria

| Target | Gram negative | | | Seq. ID |
|---|---|---|---|---|
| region | Target Genus name | Probe | Base sequence (5′ → 3′) | No. |
| ITS | Escherichia | GN-01 | CTCAGTTGGTTAGAGCGCWMCM | 3 |
| | Bacteroides | | | |
| | Haemophilus | GN-02 | YRGKYCTRTAGCTCAGTTGGTT | 4 |
| | Klebsiella | | | |
| | Enterobacter | GN-03 | TGAGAWRTTTGCTCTTTAAMAA | 5 |
| | Pseudomonas | | | |
| | Serratia | GN-04 | ATTGTCTTATGTTCTTTAAAAA | 6 |

 Mixed Base∘| Code Name
W: A + T, M: A + C, Y: C + T, R: A + G, K: G + T

New oligonucleotides for probes detecting genus-specific sepsis-causing bacteria developed in the present invention are as shown in Table 3.

TABLE 3

New probes for bacteria genus-specific detection
of sepsis-causing bacteria

| | | | | |
|---|---|---|---|---|
| Target | | | | Seq. ID |
| region | Genus name | Probe | Base sequence (5′ → 3′) | No. |

| | | | | |
|---|---|---|---|---|
| ITS | Bacteroides | Bact-01 | ACACTGATAATGTAGAGGTCG | 7 |
| | | Bact-02 | GTTCGAATCCGTTATTCTCC | 8 |
| | | Bact-03 | GTAGAGGTCGGCAGTTC | 9 |
| | | Bact-04 | GAGCGCTACACTGATAATG | 10 |
| | Enterococcus | Entc-01 | GAGTTTTTTAATAAGTTCAATTG | 11 |
| | | Entc-02 | CGCGTTGAATGAGTTTTTTAATA | 12 |
| | Haemophilus | Hae-01 | CGAATCCCCGTGGGGACGCCA | 13 |
| | Klebsiella | Kleb-01 | AAAGAACCTGCCTTTGTAGTG | 14 |
| | | Kleb-02 | TGAAAATTGAAACGACACACAG | 15 |
| | | Kleb-03 | AACCTGCCTTTGTAGTG | 16 |
| | Klebsiella/ | Kleb/Entb- | TAATGTGTGTTCGAGTCTCT | 17 |
| | Enterobacter | 01 | | |
| | Listeria | Lis-01 | GTTCTTTGAAAACTAGATAA | 18 |
| | | Lis-02 | GAAAGTTAGTAAAGTTAGCA | 19 |
| | | Lis-03 | CACAAGTAACCGAGAATCATCTG | 20 |
| | Pseudomonas | Pse-01 | CCCACACGAATTGCTTGATTC | 21 |
| | | Pse-02 | TTTCGGCGAATGTCGTCTTC | 22 |
| | | Pse-03 | ACAGTATAACCAGATTGCTT | 23 |
| | | Pse-04 | CAGATTGCTTGGGGTTATAT | 24 |
| | Serratia | Ser-01 | CACCTCCTTACCTAAWGATATT | 25 |
| | | Ser-02 | CCTCCTTACCTAAWGATATT | 26 |
| | Staphylococcus | Sta-01 | GACATATTGTATTCAGTTTTG | 27 |
| | | Sta-02 | AGATTTTACCAAGCAAAACCG | 28 |
| | Streptococcus | Str-01 | CATTGAAAATTGAATAWCKA | 29 |
| | | Str-02 | ATTGAATAWCKATATCAAAT | 30 |

 Mixed Base∘| Code Name
W: A + T, K: G + T

New oligonucleotides for probes detecting species-specific sepsis-causing bacteria developed in the present invention are as shown in Table 4.

TABLE 4
New probes for bacteria species-specific detection
of sepsis-causing bacteria

| | | | | |
|---|---|---|---|---|
| | | | | Seq. |
| Target | | | | ID |
| Region | Species name | Probe | Base sequence (5′ → 3′) | No. |

| | | | | |
|---|---|---|---|---|
| ITS | Bacteroides | Bact.fra-01 | GATATTTTATCTTGTATGAT | 31 |
| | fragilis | Bact.fra-02 | TCCGATACCGCGACCTAAC | 32 |
| | | Bact.fra-03 | GGCTTGCGTGTAATCAAACG | 33 |
| | | Bact.fra-04 | GAAAAGGAGATGAATCTGGC | 34 |
| | Bacteroides | Bact.the-01 | CTAACATCAGGTAGACAAGG | 35 |
| | thetaiotaomicron | Bact.the-02 | CTCCGAACAAAAATTGTCGT | 36 |
| | | Bact.the-03 | GGTTAATACCTGATACTT | 37 |
| | Bacteroides | Bact.ova-01 | TGTCGTAACTGTCACAAACG | 38 |
| | ovatus | Bact.ova-02 | TGACAGCCACTTAAAGACTT | 39 |
| | | Bact.ova-03 | CCAGTATGAGAATAAAACGTT | 40 |
| | Enterococcus | Entc.faeca-01 | CGTCTTTACTTTGTTCAGT | 41 |
| | faecalis | Entc.faeca-02 | GCTTATTTATTGATTAACC | 42 |
| | Enterococcus | Entc.faeci-01 | AGACTACACAATTTGTTTTT | 43 |
| | faecium | Entc.faeci-02 | CTTGATCTAACTTCTATCGC | 44 |
| | Enterococcus | Entc.avi-01 | CATGGGAATTAGTAAGACCC | 45 |
| | avium | Entc.avi-02 | ACTAAGGAACATGATCGCT | 46 |
| | | Entc.avi-03 | GGATACAGAAACAATTTTAA | 47 |
| | Enterobacter | Entb.clo-01 | TGAGACTGTACGTCCCCT | 48 |
| | cloacae | Entb.clo-02 | AGTGAAAGTCACCTGCCGTC | 49 |
| | Enterobacter | Entb.aer-01 | AAGTAGAGAAGCAAGGGGTC | 50 |
| | aerogenes | Entb.aer-02 | AGTGAAAGACGCGTGCCGATAT | 51 |
| | Enterobacter | Entb.agg-01 | GATACCTTCCCGCGCAGTGTCC | 52 |
| | agglomerans | Entb.agg-02 | GTGTCTCCATACAGTATCTC | 53 |
| | | Entb.agg-03 | TGAGCAGGACGGCTGCCAAG | 54 |
| | | Entb.agg-04 | CCAAGTCGTGACACATTTGT | 55 |
| | Escherichia coli | Esc.col-01 | AAAGAAGCGTWCTTTGMAGTG | 56 |
| | | Esc.col-02 | ATCTCAAAACTCATCTTCGGG | 57 |
| | | Esc.col-03 | AAAACTCATCTTCGGGTGATG | 58 |
| | Haemophilus | Hae.inf-01 | CTTTATTAGATTGTCTTAC | 59 |
| | influenzae | Hae.inf-02 | GAGAGAAAGTCTGAGTAGGCA | 60 |
| | | Hae.inf-03 | AATCAAGTGTTTAGTTGAAT | 61 |
| | | Hae.inf-04 | GACAAGATTAAAAACGAAGCG | 62 |
| | Haemophilus | Hae.duc-01 | TAAAGACATCACAAGTACTC | 63 |
| | ducreyi | Hae.duc-02 | GATTGTTTGATTGTTTTAG | 64 |
| | | Hae.duc-03 | AAGTAGAAAGTCTGAGTAATC | 65 |
| | | Hae.duc-04 | AGCTAAGTGTTTAGTCTAAA | 66 |
| | Klebsiella | Kleb.pne-O1 | GACGCGTGCCGATGTATC | 67 |
| | pneumoniae | Kleb.pne-02 | TTGAGACTTCAGTGTCCCCT | 68 |
| | | Kleb.pne-03 | ACGCGTGCCGAWSTATCTCA | 69 |
| | Klebsiella | Kleb.oxy-01 | GCTGCGAAGTCGCGACACCT | 70 |
| | oxytoca | Kleb.oxy-02 | GCGACACGACGATGTTTTAC | 71 |
| | | Kleb.oxy-03 | GCACAACCAACCGATACCT | 72 |
| | Listeria | Lis.mon-01 | CATAGATAATTTATTATTTATGAC | 73 |
| | monocytogenes | | | |
| | Listeria | Lis.iva-01 | CACTCTCTTAGATGTCAGAT | 74 |
| | ivanovii | Lis.iva-02 | CTGTATAACCTATTTAAGGG | 75 |
| | Listeria grayi | Lis.gra-01 | GAAACTTTCCGCTTTGGAAG | 76 |
| | | Lis.gra-02 | CTTACGCAATCGCGTAAAT | 77 |
| | | Lis.gra-03 | GAAACTTGCGTTTTTCATTCT | 78 |
| | Listeria | Lis.wel-01 | CGCAAGGCTACATGCTCTGGA | 79 |
| | welshimeri | Lis.wel-02 | AGAAAACAAAATATTATTTCC | 80 |
| | | Lis.wel-03 | CTGATACCAACGATTAGAAA | 81 |
| | Pseudomonas | Pse.aeru-01 | GCTTGATTCACTGGTTAG | 82 |
| | aeruginosa | Pse.aeru-02 | CCATCTAAAACAATCGTCG | 83 |
| | Pseudomonas | Pse.stu-01 | ACCCGAGAGTAACGATTGG | 84 |
| | stutzeri | Pse.stu-02 | CCATTAACTCTAGTCGCCG | 85 |
| | | Pse.stu-03 | CACGTTATAGACAGTAACC | 86 |
| | Serratia | Ser.mar-01 | AAGATATTAGTTCGAGTGGC | 87 |
| | marcescens | Ser.mar-02 | TAGTTCGAGTGGCGTGCTCAC | 88 |
| | Serratia | Ser.gri-01 | GATATTGATTGCGTGAAGTGC | 89 |
| | grimesii | | | |
| | Serratia | Ser.ent-01 | GATATTGATTCGAGTGAAGTG | 90 |
| | entomophila | | | |
| | Staphylococcus | Sta-au-01 | TCACAAGATTAATAACGCGTTT | 91 |
| | aureus | | | |
| | Staphylococcus | Sta.epi-01 | TTGAATTCVTAAATAATCGC | 92 |
| | epidermidis | Sta.epi-02 | CTAGTTTTAGCTATTTATTT | 93 |
| | Staphylococcus | Sta.sap-01 | CTTACGAAGATGCAGGAAT | 94 |
| | saprophyticus | Sta.sap-02 | CAACCGACTTGTTCGTGTTG | 95 |
| | Streptococcus | Str-aga-01 | AGGAAACCTGCCATTTGCGTC | 96 |
| | agalactiae | Str-aga-02 | AGTTTTCTAGTTTTAAAGAAA | 97 |
| | Streptococcus | Str-pne-01 | AAGGAACTGCGCATTGGTCT | 98 |
| | pneumoniae | Str-pne-02 | ATCACCAAGTAATGCACATTG | 99 |
| | Streptococcus | Str-pyo-01 | ACACGTTTATCGTCTTATTTAG | 100 |
| | pyogenes | Str-pyo-02 | CGATCTAGAAATAGATTGTA | 101 |
| | Streptococcus | Str-bov-01 | ACGGAAGCACGTTTGGGTATTG | 102 |
| | bovis | Str-bov-02 | ATTCAAAGATTGTCCATTGA | 103 |
| | | Str-bov-03 | GTTTAAGGTCAACAGAACCAA | 104 |

 Mixed Base∘| Code Name
W: A + T, M: A + C, S: G + C, V: G + A + C

### BEST MODE FOR CARRYING OUT THE INVENTION

The present invention will be described in greater detail by means of following examples. The following examples are for illustrative purpose and are not intended to limit the scope of the invention.

### Example 1

### Incubation of Bacteria and Isolation of Genomic DNA

Total 56 kinds of strains were obtained from the American Type Culture Collection (ATCC). The strains were selected in each culturing media under each culturing conditions according to manual provided by ATCC. From the cultured media, strain colonies were obtained with a white gold ear and input 1 ml tube, 100 ul of InstaGene matrix (Bio-Rad, USA) was added thereto and suspended, and reaction was performed at 56° C. for 30 minutes in constant temperature bath. And then, the reactant was shook for 10 seconds, heated at 100° C. for 8 min, shook again for 10 sec, centrifuged at 12,000 rpm for 3 min, recovered DNA.

The strains used were as followed Table 5:

TABLE 5

| No. | Genus | Species | Gram | Source |
|---|---|---|---|---|
| 1 | Streptococcus | Streptococcus agalactiae | (+) | ATCC 13813 |
| 2 | | Streptococcus bovis | (+) | ATCC 33317 |
| 3 | | Streptococcus pneumoniae | (+) | ATCC 33400 |
| 4 | | Streptococcus pyogenes | (+) | ATCC 19615 |
| 5 | | Streptococcus mutans | (+) | ATCC 25175 |
| 6 | | Streptococcus intermedius | (+) | KCTC 3268 |
| 7 | Listeria | Listeria grayi | (+) | ATCC 19120 |
| 8 | | Listeria ivanovii subsp. ivanovii | (+) | ATCC 19119 |
| 9 | | Listeria monocytogenes | (+) | ATCC 19111 |
| 10 | | Listeria welshimeri | (+) | ATCC 35897 |
| 11 | | Listeria innocua | (+) | ATCC 33090 |
| 12 | Staphylococcus | Staphylococcus epidermidis | (+) | ATCC 12228 |
| 13 | | Staphylococcus saprophyticus | (+) | ATCC 15305 |
| 14 | | Staphylococcus aureus | (+) | ATCC 25923 |
| 15 | | Staphylococcus hominis | (+) | ATCC 27844 |
| 16 | | Staphylococcus warneri | (+) | ATCC 27836 |
| 17 | Enterococcus | Enterococcus avium | (+) | ATCC 14025 |
| 18 | | Enterococcus faecalis | (+) | ATCC 19433 |
| 19 | | Enterococcus faecium | (+) | ATCC 8043 |
| 20 | | Enterococcus flavescens | (+) | ATCC 49996 |
| 21 | | Enterococcus solitarius | (+) | ATCC 49428 |
| 22 | Escherichia | Escherichia coli | (−) | ATCC 33572 |
| 23 | | Escherichia coli | (−) | ATCC 10799 |
| 24 | | Escherichia coli | (−) | ATCC 39403 |
| 25 | | Escherichia sp. | (−) | ATCC 21073 |
| 26 | Haemophilus | Haemophilus influenzae | (−) | ATCC 19418 |
| 27 | | Haemophilus ducreyi | (−) | ATCC 33940 |
| 28 | Citrobacter | Citrobacter freundii | (−) | ATCC 33128 |
| 29 | Bacteroides | Bacteroides thetaiotaomicron | (−) | ATCC 29741 |
| 30 | | Bacteroides fragilis | (−) | ATCC 25285 |
| 31 | | Bacteroides ovatus | (−) | ATCC 8483 |
| 32 | | Bacteroides vulgatus | (−) | ATCC 29327 |
| 33 | Klebsiella | Klebsiella oxytoca | (−) | ATCC 13182 |
| 34 | | Klebsiella pneumoniae | (−) | ATCC 15380 |
| 35 | | Klebsiella aerogenes | (−) | KCTC 2619 |
| 36 | | Klebsiella planticola | (−) | ATCC 15380 |
| 37 | Pseudomonas | Pseudomonas aeruginosa | (−) | ATCC 10145 |
| 38 | | Pseudomonas stutzeri | (−) | ATCC 17588 |
| 39 | | Pseudomonas reptilivora | (−) | ATCC 14878 |
| 40 | | Pseudomonas resinovorans | (−) | ATCC 14235 |
| 41 | Enterobacter | Enterobacter aerogenes | (−) | ATCC 13048 |
| 42 | | Enterobacter agglomerans | (−) | ATCC 27987 |
| 43 | | Enterobacter cloacae | (−) | ATCC 13047 |
| 44 | | Enterobacter gergoviae | (−) | ATCC 33426 |
| 45 | | Enterobacter sakazakii | (−) | ATCC 29544 |
| 46 | Serratia | Serratia marcescens | (−) | ATCC 13880 |
| 47 | | Serratia entomophila | (−) | KCTC 2934 |
| 48 | | Serratia grimesii | (−) | ATCC 14460 |
| 49 | | Serratia ficaria | (−) | ATCC 33105 |
| 50 | | Serratia odorifera | (−) | ATCC 33077 |
| 51 | Salmonella | Salmonella bongori | (−) | ATCC 43975 |
| 52 | Acinetobacter | Acinetobacter baumannii | (−) | ATCC 19606 |
| 53 | Bacillus | Bacillus cereus | (+) | KCTC 3711 |
| 54 | Vibrio | Vibrio parahemolyticus | (−) | ATCC 17802 |
| 55 | Shigella | Shigella boydii | (−) | ATCC 8700 |
| 56 | Neisseria | Neisseria meningitidis | (−) | ATCC 13077 |

### Example 2

### Preparation of Probes

All probes used for detection of sepsis-causing bacteria is confirmed specificity of probes by multiple alignment and BLAST searching as selecting ITS target base sequence of sepsis-causing bacteria published in Genbank. In the present invention, gram positive-specific probes of sepsis-causing bacteria is only complementary in species of gram-positive bacteria, is selected from base sequence having very lower similarity to other species. And gram negative-specific probes is only complementary in species of selected gram-negative bacteria, is selected base sequence having very lower similarity to other species. Moreover, in the present invention, genus-specific probes of sepsis-causing bacteria is only complementary in species of each genus, is selected from base sequence having very lower similarity to species of other genus. And species-specific probes is only complementary in each species, is selected from base sequence having very lower similarity to species of other species. Designed gram-positive bacteria, gram-negative bacteria and genus and species-specific probes were standed for in Table 1 to Table 4. Above all probes can be used as not being limited in base sequence of Table 1 to Table 4 but being designed primers and probes consisted of base sequence comprising it.

### Example 3

### Preparation of Taget DNA

To amplication of ITS target region for detecting sepsis-causing bacteria, there is used as labeling biotin respectively in common primers (forward 16S-1387F: 5′-biotin-GCC TTG TAC ACW CCG CCC-3′) (Applied and Environmental Microbiology, 64 (2), p. 795-799, 1998) of 16S rDNA having been known and common primers (backward 23S-520R: 5′-biotin-NAG MC CTG AAA CCG TGT GC-3′) (Patent No. 04-68313, SEQ ID No. 54) of 23S rDNA which the present inventor develops directly and is pending patent. PCR were carried out in follow condition. Reaction composition is added to water to be 25 ul of total volume after adding 10□ PCR buffer (100 mM KCl, 20 mM Tris HCl (pH 9.0), 15 mM MgCl₂) 5 μl, dNTP (deoxynucleoside triphosphates) mixture (dATP, dGTP, dTTP, and dCTP each 10 mM) 1 μl, forward and backward primers (each 10 pmole) each 1 μl, Taq polymerase (5 units/μl, QIAGEN, Inc., Valencia, USA) 0.2 μl, template DNA 4 μl. Reaction condition is denaturation at 94° C. for 3 minutes, denaturation at 94° C. for 1 minute. Annealing reaction is carried out at 50° C. for 1 minute, extension reaction is carried out at 72° C. for 1 minute, and we repeated 30 times these process.

### Example 4

### Probe Immobilization on Support

The probes prepared in Example 2 is hybrided by 9 Guanine bases, 3 Amine base of spacer and 15˜25 base sequence in 5′ end. The common probes which can know existence of bacteria used in experiment is directly developed by the present inventor, is used base sequence (Uni-459: 5′-CCG ATA GTG AAC CAG TAC C-3′) (Patent No. 04-68313, SEQ ID No. 46) being applying for a patent. The slide fixes all probes on surface of support using BMT Guanine Chip™ (Biomatrix Technology Co., Ltd. Korea) recognizing guanine and fixing biological molecular on support, then the slide is manufactured by washing non-fixed probes.

### Example 5

### Hybridization

The biotin-labeled target products prepared in Example 3 were thermally treated to be denaturated in to single strands and cooled to 4° C. To detect interaction PCR product with probes, there is manufactured reaction solution containing Cy5-streptavidin or Cy3-streptavidin (Amersham pharmacia biotech, USA) and 60 ml of hybridization reaction solution comprising 1˜5 ul of target DNA. This hybridization reaction solution was portioned on the slide glass after probes attachment and washing, and the slide glass was covered with a cover seal and reacted at 40° C. for 30 minutes.

### Example 6

### Unhybridized DNA Washing

To wash out unhybridized target DNAs, the cover slip was removed using a 2×SSC washing solution (300 mm NaCl, 30 mm Na-Citrate, pH 7.0), and the slide was washed with 2×SSC and then 0.2×SSC, followed by centrifugation to fully dry to the slide glass.

### Example 7

### Analysis of the Results

The hybridized result was scanned using a non-confocal laser scanner (GenePix 4000Am, Axon Instruments, USA) and analyzed by image analysis. The result is standed for follow Table 6.

TABLE 6
The result of specific hybridization reaction for the probes of the present invention

| | | | | Specific reaction probe's Seq. ID No. | | | |
|---|---|---|---|---|---|---|---|
| | | | | | | | Uni- |
| | No. | Species | Gram | G(+)/(−)probe | Genus probe | Species probe | probe |
| Sepsis | 1 | Streptococcus | (+) | Seq. ID No. | Seq. ID No. | Seq. ID No. | KR |
| causing | | agalactiae | | 1 to 2 | 29 to 30 | 96 to 97 | Patent |
| bacteria | 2 | Streptococcus | (+) | Seq. ID No. | Seq. ID No. | Seq. ID No. | application |
| | | bovis | | 1 to 2 | 29 to 30 | 102 to 104 | No. |
| | 3 | Streptococcus | (+) | Seq. ID No. | Seq. ID No. | Seq. ID No. | 04- |
| | | pneumoniae | | 1 to 2 | 29 to 30 | 98 to 99 | 68313 |
| | 4 | Streptococcus | (+) | Seq. ID No. | Seq. ID No. | Seq. ID No. | (Seq. ID |
| | | pyogenes | | 1 to 2 | 29 to 30 | 100 to 101 | No. 46) |
| | 5 | Streptococcus | (+) | Seq. ID No. | Seq. ID No. | (—)** | |
| | | mutans | | 1 to 2 | 29 to 30 | | |
| | 6 | Streptococcus | (+) | Seq. ID No. | Seq. ID No. | (—)** | |
| | | intermedius | | 1 to 2 | 29 to 30 | | |
| | 7 | Listeria grayi | (+) | Seq. ID No. | Seq. ID No. | Seq. ID No. | |
| | | | | 1 to 2 | 18 to 20 | 76 to 78 | |
| | 8 | Listeria ivanovii | (+) | Seq. ID No. | Seq. ID No. | Seq. ID No. | |
| | | | | 1 to 2 | 18 to 20 | 74 to 75 | |
| | 9 | Listeria | (+) | Seq. ID No. | Seq. ID No. | Seq. ID No. | |
| | | monocytogenes | | 1 to 2 | 18 to 20 | 73 | |
| | 10 | Listeria welshimeri | (+) | Seq. ID No. | Seq. ID No. | Seq. ID No. | |
| | | | | 1 to 2 | 18 to 20 | 79 to 81 | |
| | 11 | Listeria innocua | (+) | Seq. ID No. | Seq. ID No. | (—)** | |
| | | | | 1 to 2 | 18 to 20 | | |
| | 12 | Staphylococcus | (+) | Seq. ID No. | Seq. ID No. | Seq. ID No. | |
| | | epidermidis | | 1 to 2 | 27 to 28 | 92 to 93 | |
| | 13 | Staphylococcus | (+) | Seq. ID No. | Seq. ID No. | Seq. ID No. | |
| | | saprophyticus | | 1 to 2 | 27 to 28 | 94 to 95 | |
| | 14 | Staphylococcus | (+) | Seq. ID No. | Seq. ID No. | Seq. ID No. | |
| | | aureus | | 1 to 2 | 27 to 28 | 91 | |
| | 15 | Staphylococcus | (+) | Seq. ID No. | Seq. ID No. | (—)** | |
| | | hominis | | 1 to 2 | 27 to 28 | | |
| | 16 | Staphylococcus | (+) | Seq. ID No. | Seq. ID No. | (—)** | |
| | | warneri | | 1 to 2 | 27 to 28 | | |
| | 17 | Enterococcus avium | (+) | Seq. ID No. | Seq. ID No. | Seq. ID No. | |
| | | | | 1 to 2 | 11 to 12 | 45 to 47 | |
| | 18 | Enterococcus | (+) | Seq. ID No. | Seq. ID No. | Seq. ID No. | |
| | | faecalis | | 1 to 2 | 11 to 12 | 41 to 42 | |
| | 19 | Enterococcus | (+) | Seq. ID No. | Seq. ID No. | Seq. ID No. | |
| | | faecium | | 1 to 2 | 11 to 12 | 43 to 44 | |
| | 20 | Enterococcus | (+) | Seq. ID No. | Seq. ID No. | (—)** | |
| | | flavescens | | 1 to 2 | 11 to 12 | | |
| | 21 | Enterococcus | (+) | Seq. ID No. | Seq. ID No. | (—)** | |
| | | solitarius | | 1 to 2 | 11 to 12 | | |
| | 22 | Escherichia coli | (−) | Seq. ID No. | (—)* | Seq. ID No. | |
| | | | | 3 to 6 | | 56 to 58 | |
| | 23 | Escherichia sp. | (−) | Seq. ID No. | (—)* | (—)** | |
| | | | | 3 to 6 | | | |
| | 24 | Haemophilus | (−) | Seq. ID No. | Seq. ID No. | Seq. ID No. | |
| | | influenzae | | 3 to 6 | 13 | 59 to 62 | |
| | 25 | Haemophilus ducreyi | (−) | Seq. ID No. | Seq. ID No. | Seq. ID No. | |
| | | | | 3 to 6 | 13 | 63 to 66 | |
| | 26 | Citrobacter freundii | (−) | Seq. ID No. | (—)* | (—)** | |
| | | | | 3 to 6 | | | |
| | 27 | Bacteroides | (−) | Seq. ID No. | Seq. ID No. | Seq. ID No. | |
| | | thetaiotaomicron | | 3 to 6 | 7 to 10 | 35 to 37 | |
| | 28 | Bacteroides fragilis | (−) | Seq. ID No. | Seq. ID No. | Seq. ID No. | |
| | | | | 3 to 6 | 7 to 10 | 35 to 34 | |
| | 29 | Bacteroides ovatus | (−) | Seq. ID No. | Seq. ID No. | Seq. ID No. | |
| | | | | 3 to 6 | 7 to 10 | 38 to 40 | |
| | 30 | Bacteroides vulgatus | (−) | Seq. ID No. | Seq. ID No. | (—)** | |
| | | | | 3 to 6 | 7 to 10 | | |
| | 31 | Klebsiella oxytoca | (−) | Seq. ID No. | Seq. ID No. | Seq. ID No. | |
| | | | | 3 to 6 | 14 to 17 | 70 to 72 | |
| | 32 | Klebsiella | (−) | Seq. ID No. | Seq. ID No. | Seq. ID No. | |
| | | pneumoniae | | 3 to 6 | 14 to 17 | 67 to 69 | |
| | 33 | Klebsiella aerogenes | (−) | Seq. ID No. | Seq. ID No. | (—)** | |
| | | | | 3 to 6 | 14 to 17 | | |
| | 34 | Klebsiella planticola | (−) | Seq. ID No. | Seq. ID No. | (—)** | |
| | | | | 3 to 6 | 14 to 17 | | |
| | 35 | Pseudomonas | (−) | Seq. ID No. | Seq. ID No. | Seq. ID No. | |
| | | aeruginosa | | 3 to 6 | 21 to 24 | 82 to 83 | |
| | 36 | Pseudomonas | (−) | Seq. ID No. | Seq. ID No. | Seq. ID No. | |
| | | stutzeri | | 3 to 6 | 21 to 24 | 84 to 86 | |
| | 37 | Pseudomonas | (−) | Seq. ID No. | Seq. ID No. | (—)** | |
| | | reptilivora | | 3 to 6 | 21 to 24 | | |
| | 38 | Pseudomonas | (−) | Seq. ID No. | Seq. ID No. | (—)** | |
| | | resinovorans | | 3 to 6 | 21 to 24 | | |
| | 39 | Enterobacter | (−) | Seq. ID No. | Seq. ID No. | Seq. ID No. | |
| | | aerogenes | | 3 to 6 | 17 | 50 to 51 | |
| | 40 | Enterobacter | (−) | Seq. ID No. | Seq. ID No. | Seq. ID No. | |
| | | agglomerans | | 3 to 6 | 17 | 52 to 55 | |
| | 41 | Enterobacter cloacae | (−) | Seq. ID No. | Seq. ID No. | Seq. ID No. | |
| | | | | 3 to 6 | 17 | 48 to 49 | |
| | 42 | Enterobacter | (−) | Seq. ID No. | Seq. ID No. | (—)** | |
| | | gergoviae | | 3 to 6 | 17 | | |
| | 43 | Enterobacter | (−) | Seq. ID No. | Seq. ID No. | (—)** | |
| | | sakazakii | | 3 to 6 | 17 | | |
| | 44 | Serratia marcescens | (−) | Seq. ID No. | Seq. ID No. | Seq. ID No. | |
| | | | | 3 to 6 | 25 to 26 | 87 to 88 | |
| | 45 | Serratia entomophila | (−) | Seq. ID No. | Seq. ID No. | Seq. ID No. | |
| | | | | 3 to 6 | 25 to 26 | 90 | |
| | 46 | Serratia grimesii | (−) | Seq. ID No. | Seq. ID No. | Seq. ID No. | |
| | | | | 3 to 6 | 25 to 26 | 89 | |
| | 47 | Serratia ficaria | (−) | Seq. ID No. | Seq. ID No. | (—)** | |
| | | | | 3 to 6 | 25 to 26 | | |
| | 48 | Serratia odorifera | (−) | Seq. ID No. | Seq. ID No. | (—)** | |
| | | | | 3 to 6 | 25 to 26 | | |
| Sepsis | 49 | Salmonella bongori | (−) | Seq. ID No. | (—)* | (—)** | |
| non- | | | | 3 to 6 | | | |
| related | 50 | Acinetobacter | (−) | Seq. ID No. | (—)* | (—)** | |
| bacteria | | baumannii | | 3 to 6 | | | |
| | 51 | Bacillus cereus | (+) | Seq. ID No. | (—)* | (—)** | |
| | | | | 1 to 2 | | | |
| | 52 | Vibrio | (−) | Seq. ID No. | (—)* | (—)** | |
| | | parahemolyticus | | 3 to 6 | | | |
| | 53 | Shigella boydii | (−) | Seq. ID No. | (—)* | (—)** | |
| | | | | 3 to 6 | | | |
| | 54 | Neisseria | (−) | Seq. ID No. | (—)* | (—)** | |
| | | meningitidis | | 3 to 6 | | | |

(—)* The results of non-hybridization reaction non-comprising genus-specific probes design of the present invention
(—)** The results of non-hybridization reaction non-comprising species-specific probes design of the present invention

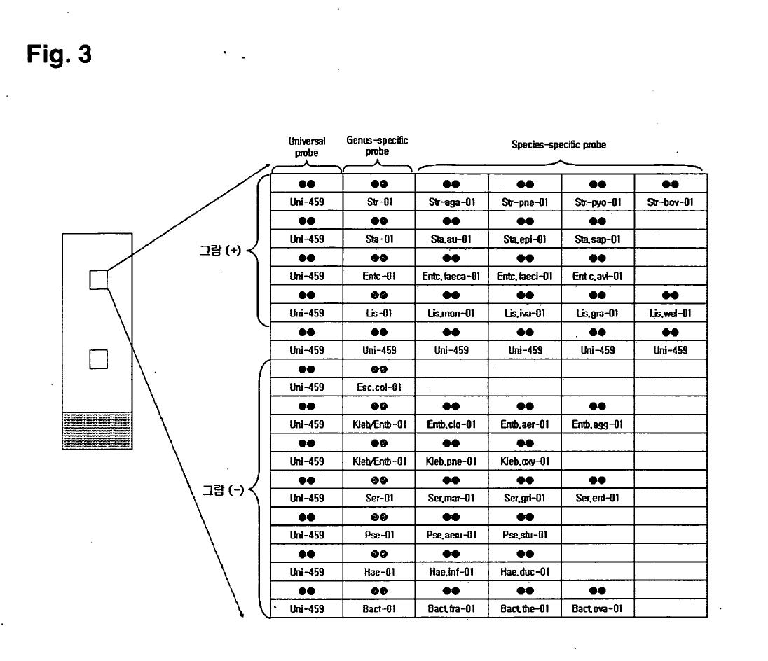

FIG. 3 show a microarray comprising a support, as a probe set, consisting of bacteria universal probes for detecting bacteria associated with sepsis and genus-specific and species-specific probes.

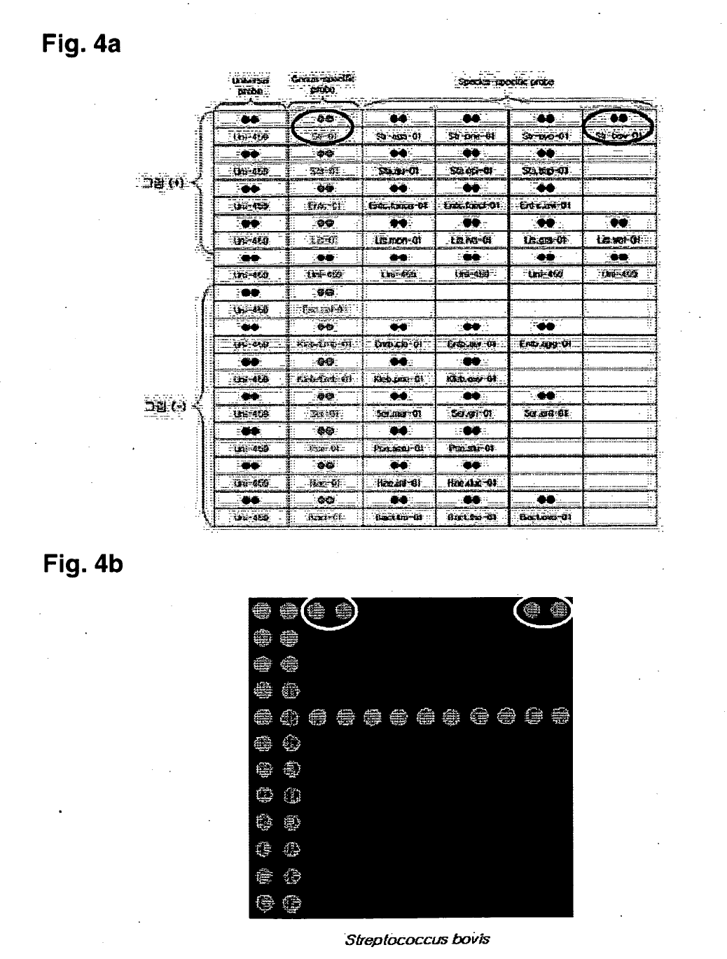

FIG. 4 shows results of hybridization reaction for Streptococcus bovis using microarray, and shows scan image of bacteria common probes (Patent No. 04-68313 SEQ ID NO. 46) and Streptococcus genus-specific (SEQ ID No. 29) and Streptococcus bovis species-specific probes (SEQ ID No. 102).

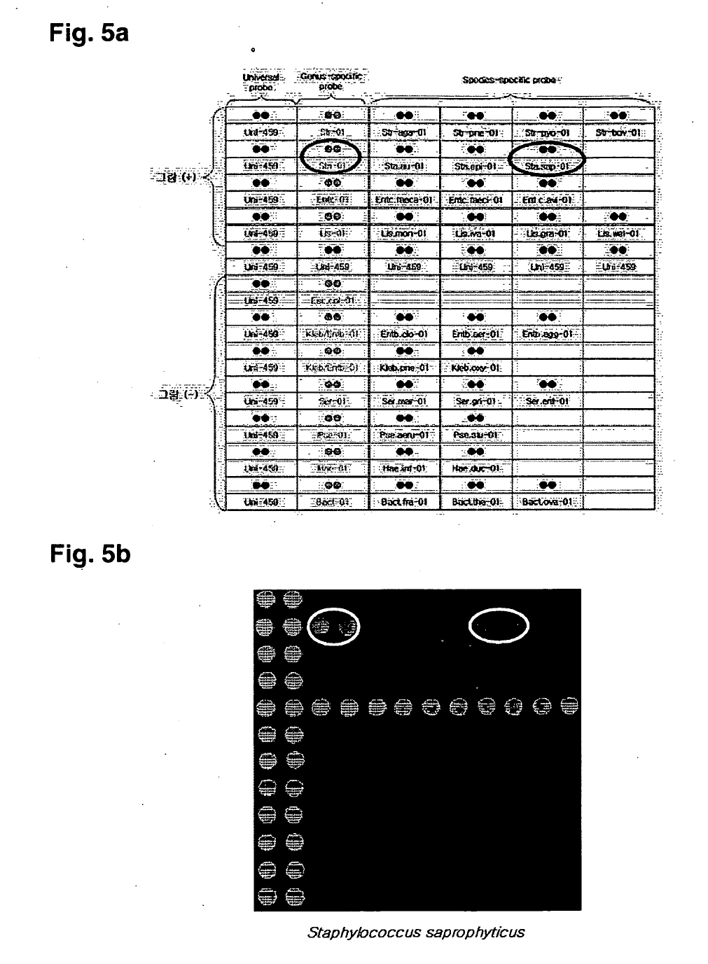

FIG. 5 shows results of hybridization reaction for Staphylococcus saprophyticus using microarray, and shows scan image of bacteria common probes (Patent No. 04-68313 SEQ ID NO. 46) and Staphylococcus genus-specific (SEQ ID No. 27) and Staphylococcus saprophyticus species-specific (SEQ ID No. 94) probes.

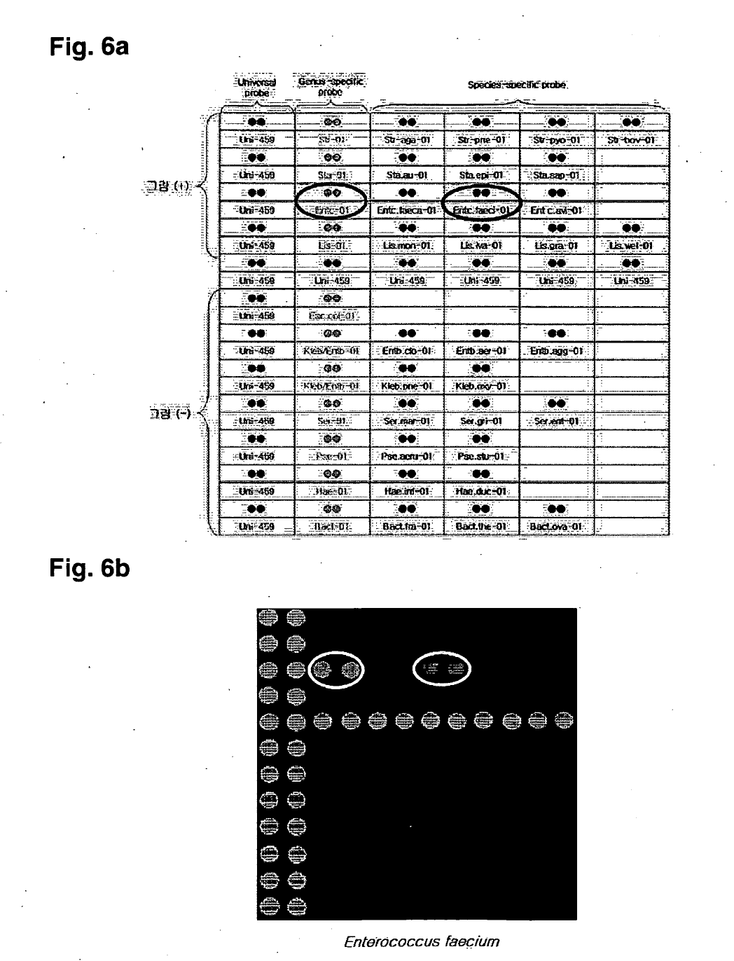

FIG. 6 shows results of hybridization reaction for Enterococcus faecium using microarray, and shows scan image of bacteria common probes (Patent No. 04-68313 SEQ ID NO. 46) and Enterococcus genus-specific (SEQ ID No. 11) and Enterococcus faecium species-specific (SEQ ID No. 43) probes.

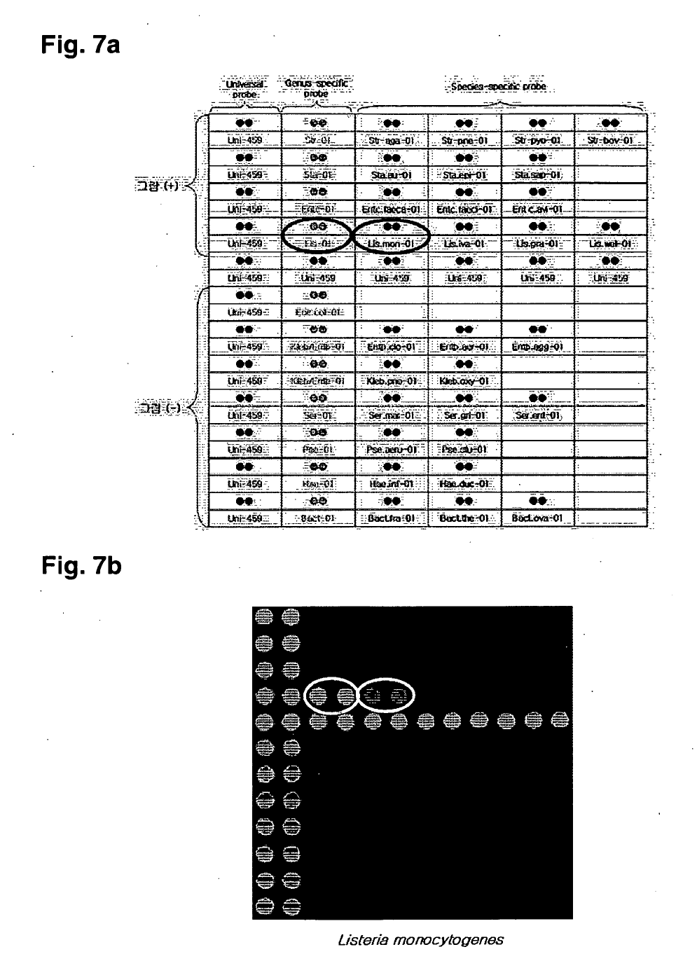

FIG. 7 shows results of hybridization reaction for Listeria monocytogenes using microarray, and shows scan image of bacteria common probes (Patent No. 04-68313 SEQ ID NO. 46) and Listeria genus-specific (SEQ ID No. 18) and Listeria monocytogenes species-specific (SEQ ID No. 73) probes.

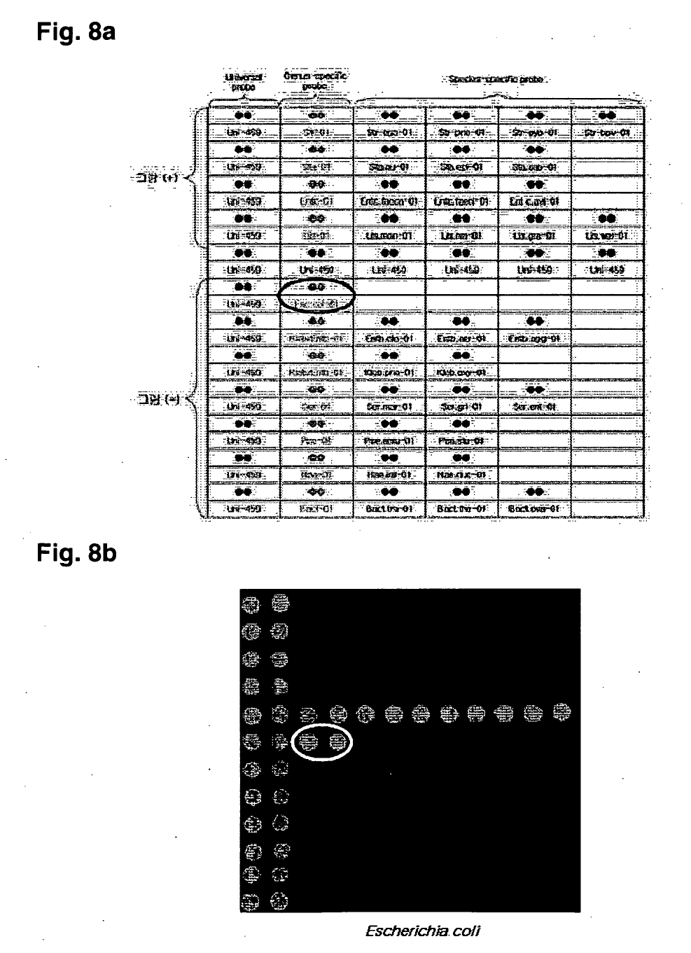

FIG. 8 shows results of hybridization reaction for Escherichia coli using microarray, and shows scan image of bacteria common probes (Patent No. 04-68313 SEQ ID NO. 46) and Escherichia genus-specific (SEQ ID No. 46) and Escherichia coli species-specific (SEQ ID No. 56) probes.

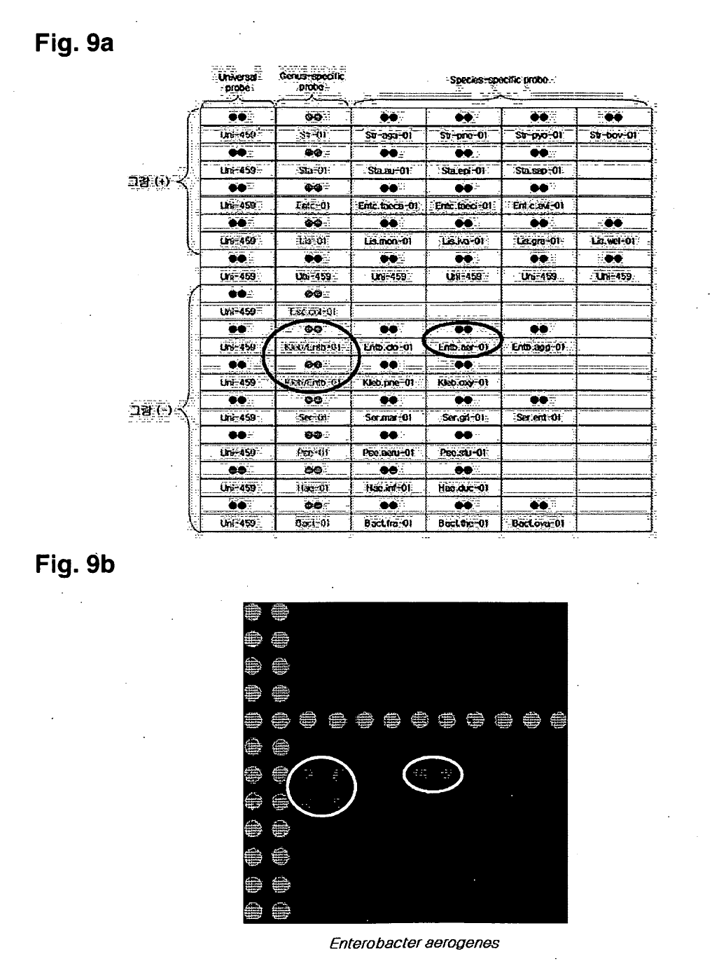

FIG. 9 shows results of hybridization reaction for Enterobacter aerogenes using microarray, and shows scan image of bacteria common probes (Patent No. 04-68313 SEQ ID NO. 46) and Enterobacter genus-specific (SEQ ID No. 17) and Enterobacter aerogenes species-specific (SEQ ID No. 50) probes.

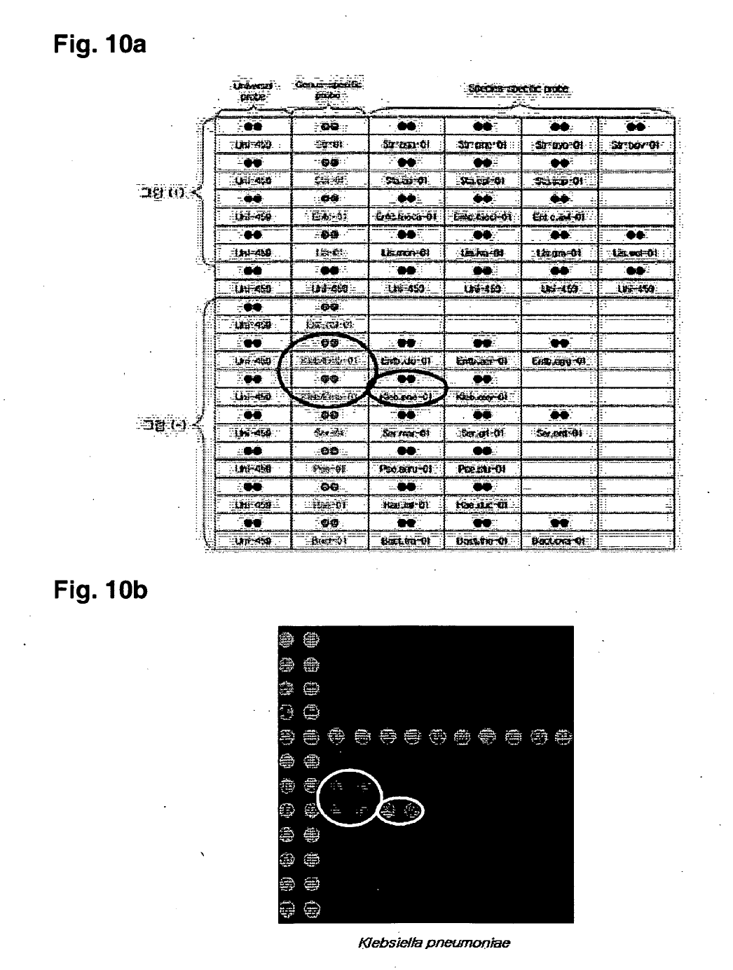

FIG. 10 shows results of hybridization reaction for Klebsiella pneumoniae using microarray, and shows scan image of bacteria common probes (Patent No. 04-68313 SEQ ID NO. 46) and Klebsiella genus-specific (SEQ ID No. 17) and Klebsiella pneumoniae species-specific (SEQ-ID No. 67) probes.

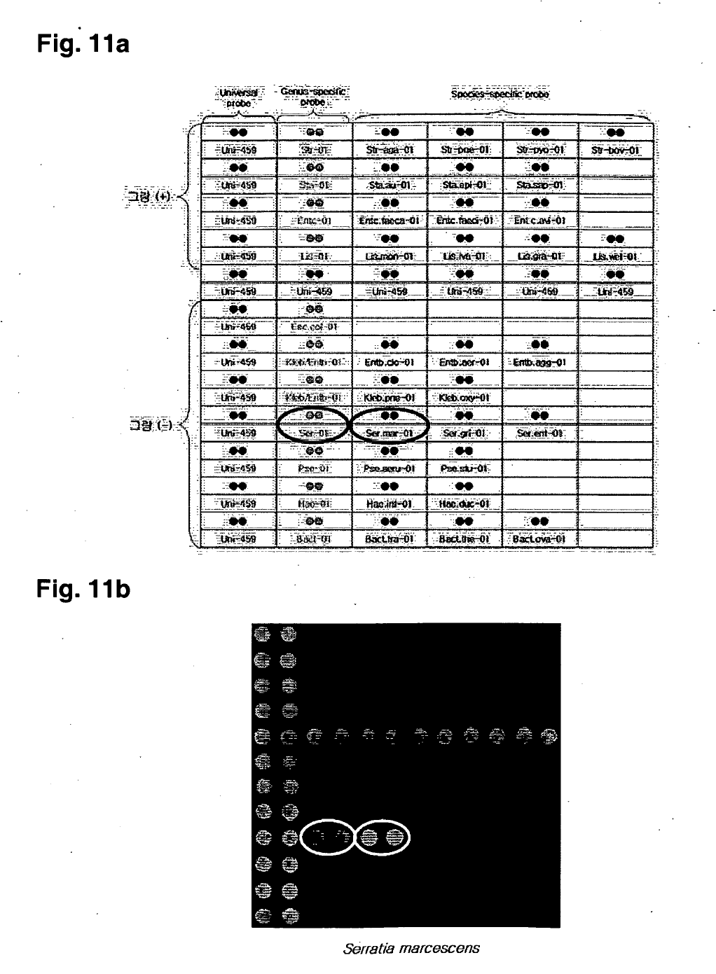

FIG. 11 shows results of hybridization reaction for Serratia marcescens using microarray, and shows scan image of bacteria common probes (Patent No. 04-68313 SEQ ID NO. 46) and Serratia genus-specific (SEQ ID No. 25) and Serratia marcescens species-specific (SEQ ID No. 87) probes.

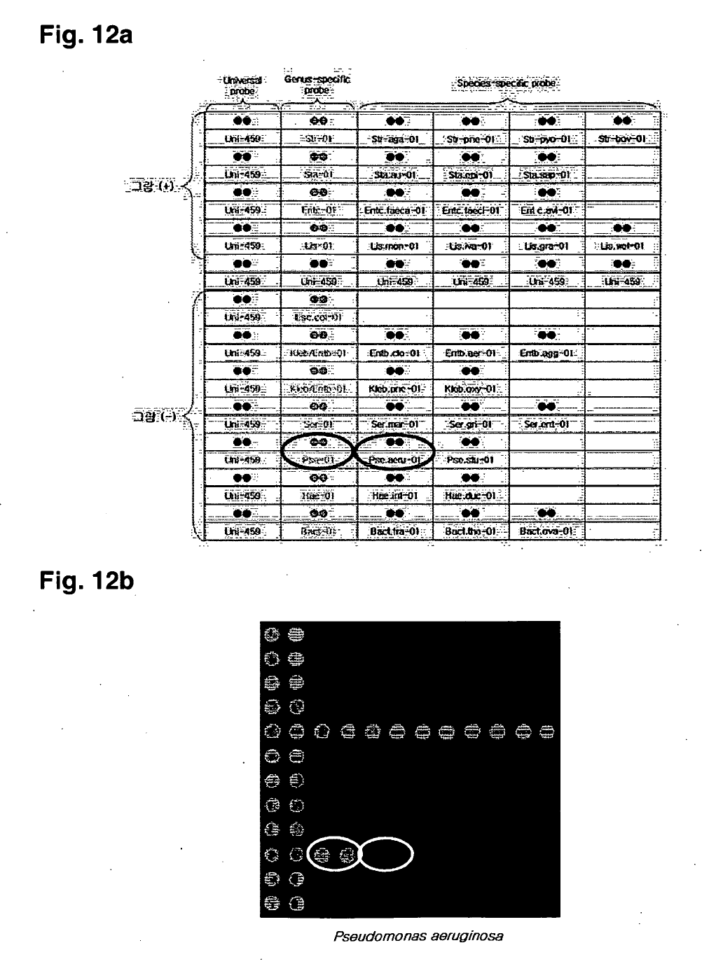

FIG. 12 shows results of hybridization reaction for Pseudomonas aeruginosa using microarray, and shows scan image of bacteria common probes (Patent No. 04-68313 SEQ ID NO. 46) and Pseudomonas genus-specific (SEQ ID No. 21) and Pseudomonas aeruginosa species-specific (SEQ ID No.82) probes.

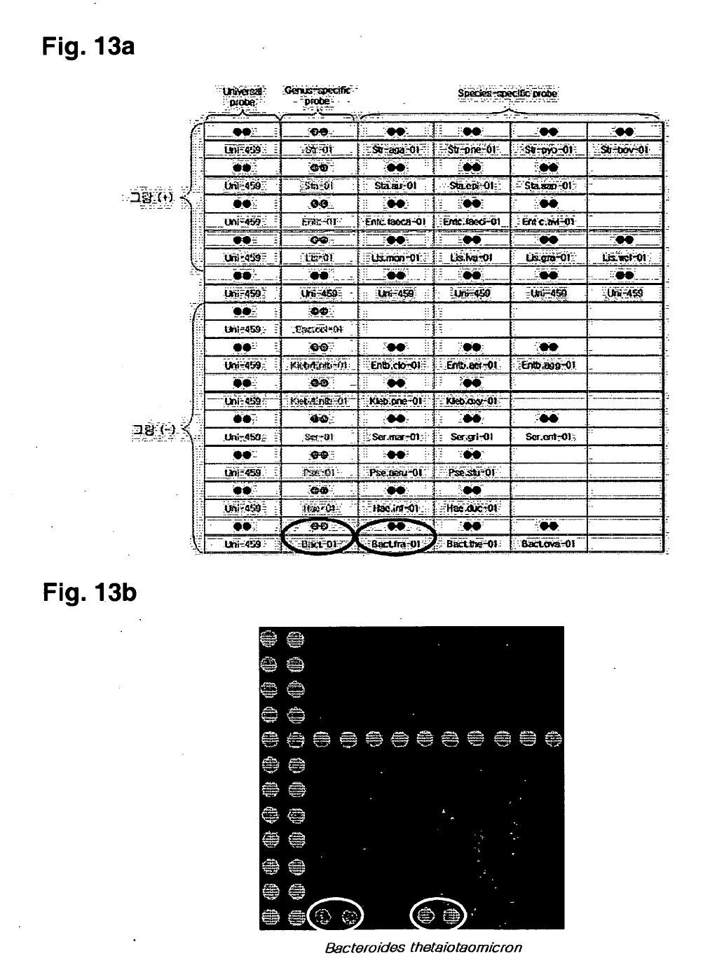

FIG. 13 shows results of hybridization reaction for Bacteroides thetaiotaomicron using microarray, and shows scan image of bacteria common probes (Patent No. 04-68313 SEQ ID NO. 46) and Bacteroides genus-specific (SEQ ID No. 7) and Bacteroides thetaiotaomicron species-specific (SEQ ID No.35) probes.

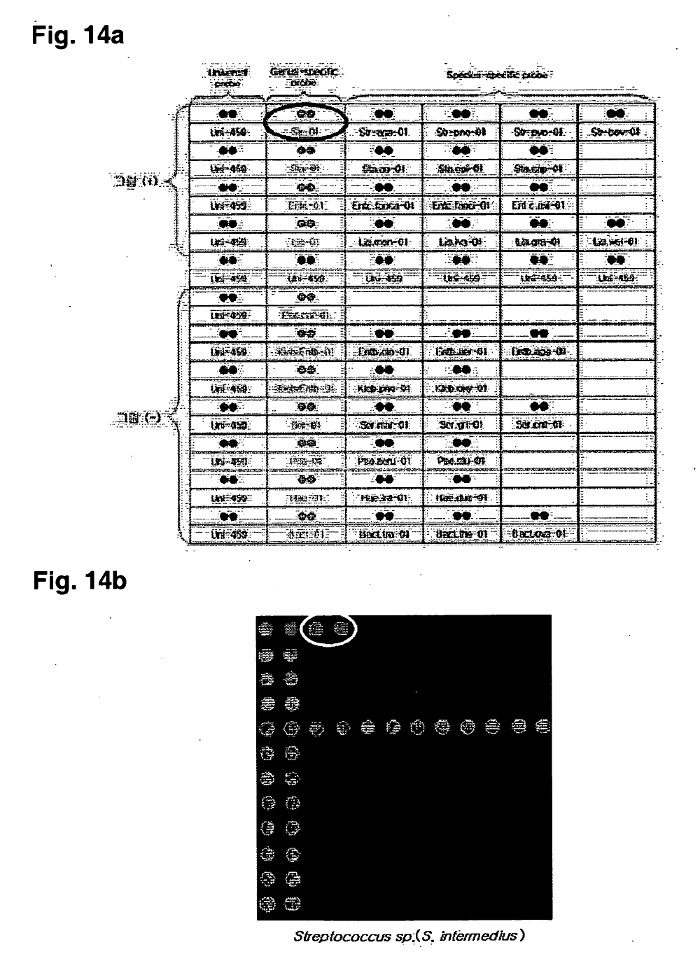

FIG. 14 shows, even though bacteria unrelated with sepsis, results of hybridization reaction for Streptococcus intermedius using microarray, and shows scan image of bacteria common probes (Patent No. 04-68313 SEQ ID NO. 46) and Streptococcus genus-specific (SEQ ID No. 46) and Streptococcus intermedius species-specific (SEQ ID No.29) probes.

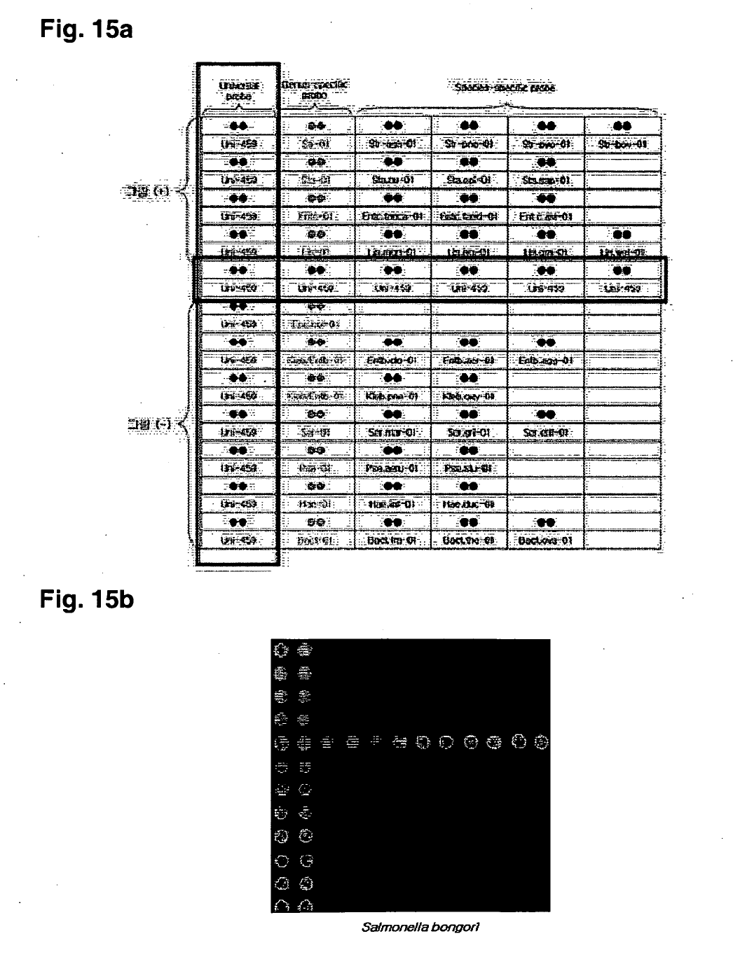

FIG. 15 shows, even though pathogenic bacteria unrelated with sepsis, results of hybridization reaction for Salmonella bongori using microarray, and shows scan image reacting to bacteria common probes (Patent No.04-68313 SEQ ID NO.46) and not reacting to specific probes for detection of sepsis-causing bacteria.

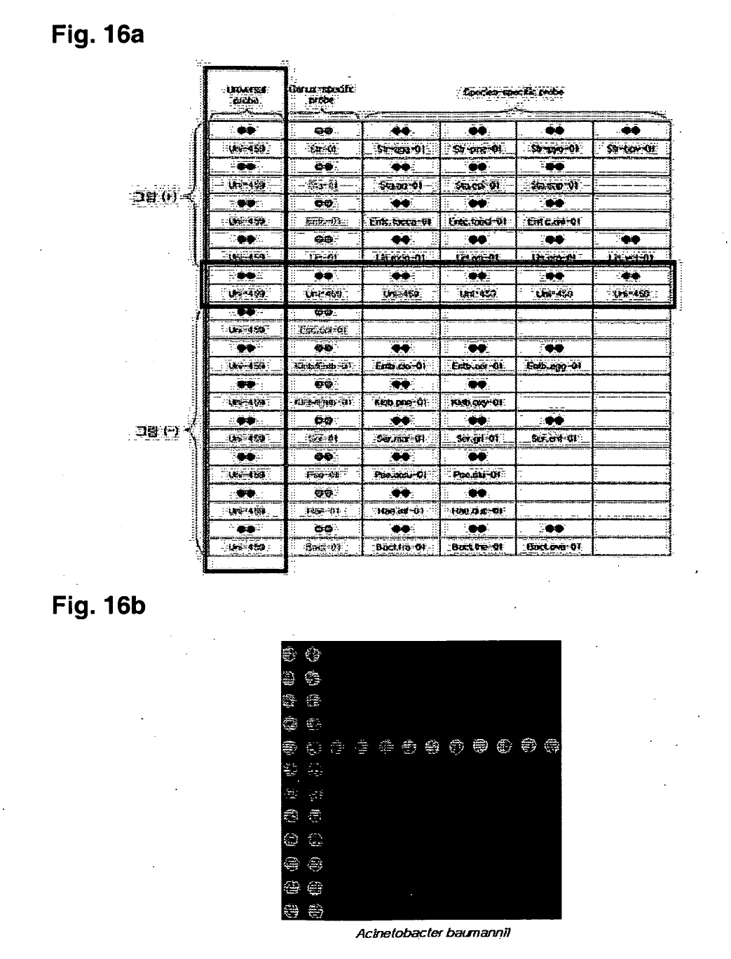

FIG. 16 shows, even though pathogenic bacteria unrelated with sepsis, results of hybridization reaction for Acinetobacter baumannii using microarray, and shows scan image reacting to bacteria common probes (Patent No.04-68313 SEQ ID NO. 46) and not reacting to specific probes for detection of sepsis-causing bacteria.

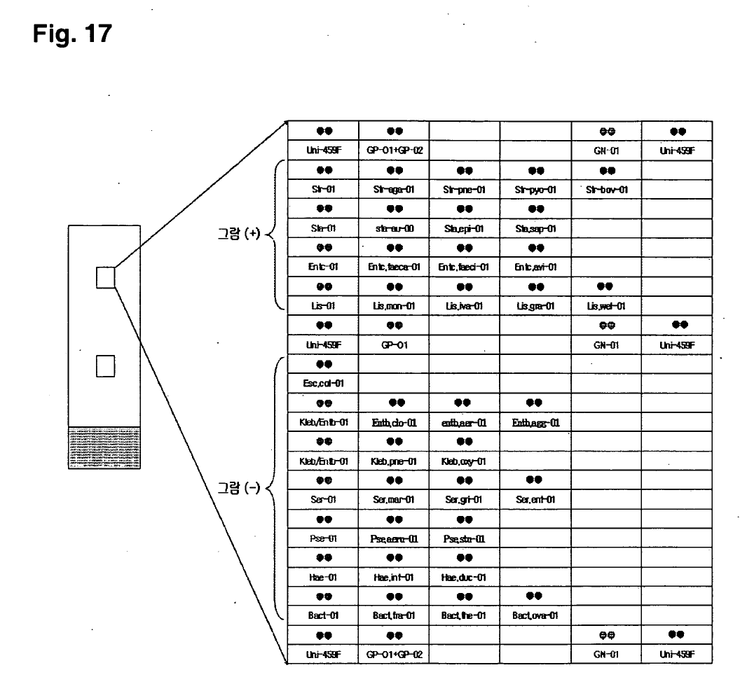

FIG. 17 shows detection for the existence of sepsis-causing bacteria, and shows microarray comprising a support, as a probe set, consisting of bacteria common probes, gram-positive and gram-negative probes and genus-specific and species-specific probes.

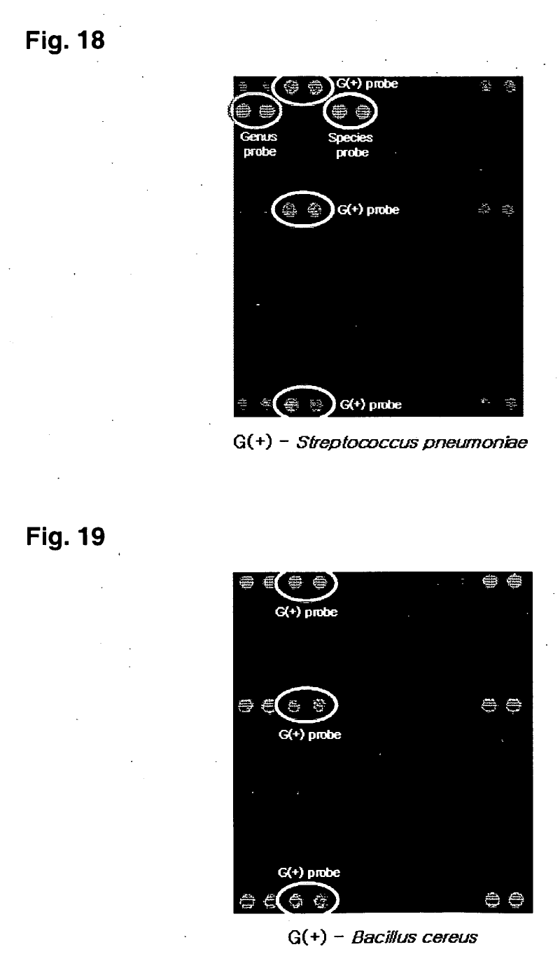

FIG. 18 shows results of hybridization reaction for Streptococcus pneumoniae of gram-positive bacteria among sepsis-causing bacteria using microarray, and shows scan image of bacteria common probes (Patent No. 04-68313 SEQ ID No. 46), gram-positive probes (SEQ ID No. 1-2) and Streptococcus genus-specific (SEQ ID No. 29) and Streptococcus pneumoniae species-specific (SEQ ID No. 98) probes.

FIG. 19 shows results of hybridization reaction for Bacillus cereus of gram-positive bacteria among bacteria unrelated with sepsis using microarray, and shows scan image reacting to bacteria common probes (Patent No. 04-68313 SEQ ID NO. 46) and gram-positive specific probes (SEQ ID No.1-2) and not reacting to specific probes for detection of sepsis-causing bacteria.

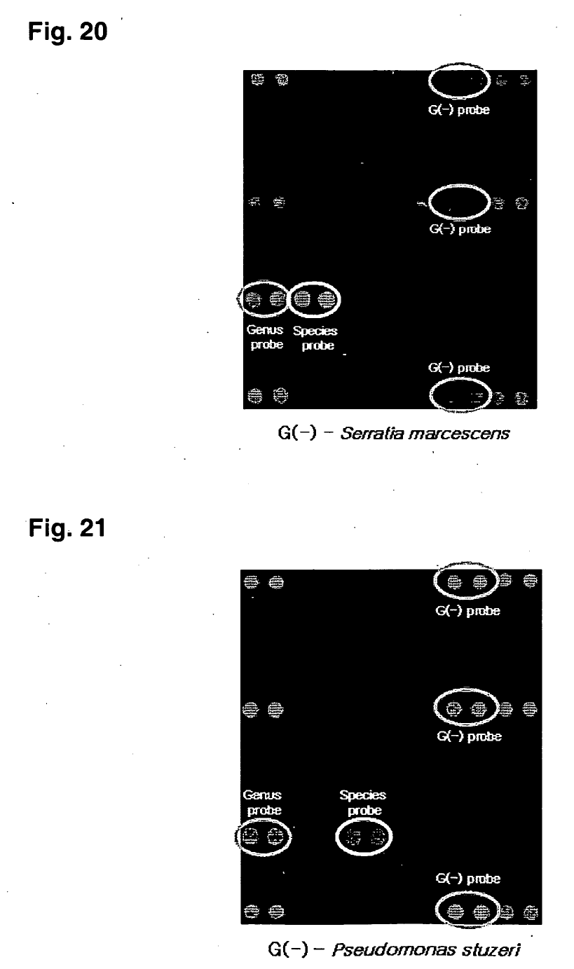

FIG. 20 shows results of hybridization reaction for Serratia marcescens of gram-negative bacteria among sepsis-causing bacteria using microarray, and shows scan image reacting to bacteria common probes (Patent No. 04-68313 SEQ ID NO. 46), gram-negative specific probes (SEQ ID No. 3), Serratia genus-specific (SEQ ID No.25) and Serratia marcescens species-specific (SEQ ID No. 87) probes.

FIG. 21 shows results of hybridization reaction for Pseudomonas stuzeri of gram-negative bacteria among sepsis-causing bacteria using microarray, and shows scan image reacting to bacteria common probes (Patent No. 04-68313 SEQ ID NO. 46), gram-negative specific probes (SEQ ID No. 3), Pseudomonas genus-specific (SEQ ID No. 21) and Pseudomonas stuzeri species-specific (SEQ ID No. 84) probes.

The composition and layout of each probes can be changed because this is only typical example of probes layout among new oligonucleotides designed in the present invention.

### INDUSTRIAL APPLICABILITY

As described above, the present invention developed the microarray and the diagnosis kit for detecting sepsis-causing bacteria comprising any one selected from a group consisting of gram positive- and gram negative-specific and genus- and species-specific probes designed from ITS of base sequence hypervariable region of the bacteria, and confirmed its specificity.

According to the present invention, the present invention can provide an antibiotics therapy for accurately removing infectious agent related to sepsis by detecting existence of sepsis-causing bacteria and identifying gram positive- and gram negative-bacteria and genus and species of the bacteria, at once. And, the present invention can prevent a patient from abuse and misuse of antibiotics and decrease time of hospital treatment and medical cost of the patient. Further, the present invention has advantage of preventing complications and reducing mortality rate.

1. An oligonucleotide for gram positive-specific detection of sepsis-causing bacteria, comprising any one base sequence selected from SEQ ID Nos. 1 to 2 or its complementary sequence.

2. An oligonucleotide for gram negative-specific detection of sepsis-causing bacteria, comprising any one base sequence selected from SEQ ID Nos. 3 to 6 or its complementary sequence.

3. An oligonucleotide for genus-specific detection of sepsis-causing bacteria, comprising any one base sequence selected from SEQ ID Nos. 7 to 30 or its complementary sequence.

4. An oligonucleotide for species-specific detection of sepsis-causing bacteria, comprising any one base sequence selected from SEQ ID Nos. 31 to 104 or its complementary sequence.

5. A primer set for amplification of sepsis-causing bacteria comprising more than one oligonucleotide according to claim 1.

6-19. (canceled)

20. A primer set for amplification of sepsis-causing bacteria comprising more than one oligonucleotide according to claim 2.

21. A primer set for amplification of sepsis-causing bacteria comprising more than one oligonucleotide according to claim 3.

22. A primer set for amplification of sepsis-causing bacteria comprising more than one oligonucleotide according to claim 4.

23. A probe set for detection of sepsis-causing bacteria comprising more than one oligonucleotide according to claim 2.

24. A probe set for detection of sepsis-causing bacteria comprising more than one oligonucleotide according to claim 3.

25. A probe set for detection of sepsis-causing bacteria comprising more than one oligonucleotide according to claim 4.

26. A kit for diagnosis of sepsis-causing bacteria comprising more than one according to claim 2.

27. A kit for diagnosis of sepsis-causing bacteria comprising more than one according to claim 3.

28. A kit for diagnosis of sepsis-causing bacteria comprising more than one according to claim 4.

29. A PCR kit comprising an oligonucleotide for gram positive-specific detection of sepsis-causing bacteria according to claim 1, as a primer set.

30. A PCR kit comprising an oligonucleotide for gram negative-specific detection of sepsis-causing bacteria according to claim 2, as a primer set.

31. A PCR kit comprising an oligonucleotide for genus-specific detection of sepsis-causing bacteria according to claim 3, as a primer set.

32. A PCR kit comprising an oligonucleotide for species-specific detection of sepsis-causing bacteria according to claim 4, as a primer set.

33. A microarray comprising an oligonucleotide for gram positive-specific detection of sepsis-causing bacteria according to claim 1, as a probe attached on a support.

34. A microarray comprising an oligonucleotide for gram negative-specific detection of sepsis-causing bacteria according to claim 2, as a probe attached on a support.

35. A microarray comprising an oligonucleotide for genus-specific detection of sepsis-causing bacteria according to claim 3, as a probe attached on a support.

36. A microarray comprising an oligonucleotide for species-specific detection of sepsis-causing bacteria according to claim 4, as a probe attached on a support.

37. The microarray according to claim 33, wherein the probe is any one selected from a group consisting of DNA (deoxyribonucleic acid), RNA (ribonucleic acid), PNA (peptide nucleic acid), LNA (Locked nucleic acid) and HNA (dihexitol nucleic acid).

38. The microarray according to claim 34, wherein the probe is any one selected from a group consisting of DNA (deoxyribonucleic acid), RNA (ribonucleic acid), PNA (peptide nucleic acid), LNA (Locked nucleic acid) and HNA (dihexitol nucleic acid).

39. The microarray according to claim 35, wherein the probe is any one selected from a group consisting of DNA (deoxyribonucleic acid), RNA (ribonucleic acid), PNA (peptide nucleic acid), LNA (Locked nucleic acid) and HNA (dihexitol nucleic acid).

40. The microarray according to claim 36, wherein the probe is any one selected from a group consisting of DNA (deoxyribonucleic acid), RNA (ribonucleic acid), PNA (peptide nucleic acid), LNA (Locked nucleic acid) and HNA (dihexitol nucleic acid).

41. The microarray according to claim 33, wherein the support is made of any one selected from a group consisting of slide glass, plastic, membrane, semi-conductive chip, silicon, gel, nano material, ceramic, metallic substance, optical fiber, and their mixture.

42. The microarray according to claim 34, wherein the support is made of any one selected from a group consisting of slide glass, plastic, membrane, semi-conductive chip, silicon, gel, nano material, ceramic, metallic substance, optical fiber, and their mixture.

43. The microarray according to claim 35, wherein the support is made of any one selected from a group consisting of slide glass, plastic, membrane, semi-conductive chip, silicon, gel, nano material, ceramic, metallic substance, optical fiber, and their mixture.

44. The microarray according to claim 36, wherein the support is made of any one selected from a group consisting of slide glass, plastic, membrane, semi-conductive chip, silicon, gel, nano material, ceramic, metallic substance, optical fiber, and their mixture.

45. A method for identification and detection of sepsis-causing bacteria, comprising the following steps:
a) purifying nucleic acids from a sample;
b) amplifying target DNAs among the purified nucleic acid;
c) hybridizing the amplified target DNA with probes of the microarray according to claim 33; and
d) detecting signals generated from the formed hybrid.

46. A method for identification and detection of sepsis-causing bacteria, comprising the following steps:
a) purifying nucleic acids from a sample;
b) amplifying target DNAs among the purified nucleic acid;
c) hybridizing the amplified target DNA with probes of the microarray according to claim 34; and
d) detecting signals generated from the formed hybrid.

47. A method for identification and detection of sepsis-causing bacteria, comprising the following steps:
a) purifying nucleic acids from a sample;
b) amplifying target DNAs among the purified nucleic acid;
c) hybridizing the amplified target DNA with probes of the microarray according to claim 35; and
d) detecting signals generated from the formed hybrid.

48. A method for identification and detection of sepsis-causing bacteria, comprising the following steps:
a) purifying nucleic acids from a sample;
b) amplifying target DNAs among the purified nucleic acid;
c) hybridizing the amplified target DNA with probes of the microarray according to claim 36; and
d) detecting signals generated from the formed hybrid.

49. The method according to claim 45, wherein the identification and detection are done at once for more than one sepsis-causing bacteria species selected from a group consisting of the following members:
a) gram positive bacteria (SEQ ID Nos. 1 to 2) and gram negative bacteria (SEQ ID Nos. 3 to 6);
b) Bacteroides genus (SEQ ID Nos. 7 to 10) and Bacteriodes species (SEQ ID Nos. 31 to 40);
c) Enterococcus genus (SEQ ID Nos. 11 to 12) and Enterococcus species (SEQ ID Nos. 41 to 47);
d) Enterobacter genus (SEQ ID Nos. 17) and Enterobacter species (SEQ ID Nos. 48 to 55);
e) Escherichia coli species (SEQ ID Nos. 56 to 58);
f) Haemophilus genus (SEQ ID Nos. 13) and Haemophilus species (SEQ ID Nos. 59 to 66);
g) Klebsiella genus (SEQ ID Nos. 14 to 17) and Klebsiella species (SEQ ID Nos. 67 to 72);
h) Listeria genus (SEQ ID Nos. 18 to 20) and Listeria species (SEQ ID Nos. 73 to 81);
i) Pseudomonas genus (SEQ ID Nos. 21 to 24) and Pseudomonas species (SEQ ID Nos. 82 to 86);
j) Serratia genus (SEQ ID Nos. 25 to 26) and Serratia species (SEQ ID Nos. 87 to 90);
k) Staphylococcus genus (SEQ ID Nos. 27 to 28) and Staphylococcus species (SEQ ID Nos. 91 to 95); and
l) Streptococcus genus (SEQ ID Nos. 29 to 30) and Streptococcus species (SEQ ID Nos. 96 to 104).

50. The method according to claim 46, wherein the identification and detection are done at once for more than one sepsis-causing bacteria species selected from a group consisting of the following members:
a) gram positive bacteria (SEQ ID Nos. 1 to 2) and gram negative bacteria (SEQ ID Nos. 3 to 6);
b) Bacteroides genus (SEQ ID Nos. 7 to 10) and Bacteriodes species (SEQ ID Nos. 31 to 40);
c) Enterococcus genus (SEQ ID Nos. 11 to 12) and Enterococcus species (SEQ ID Nos. 41 to 47);
d) Enterobacter genus (SEQ ID Nos. 17) and Enterobacter species (SEQ ID Nos. 48 to 55);
e) Escherichia coli species (SEQ ID Nos. 56 to 58);
f) Haemophilus genus (SEQ ID Nos. 13) and Haemophilus species (SEQ ID Nos. 59 to 66);
g) Klebsiella genus (SEQ ID Nos. 14 to 17) and Klebsiella species (SEQ ID Nos. 67 to 72);
h) Listeria genus (SEQ ID Nos. 18 to 20) and Listeria species (SEQ ID Nos. 73 to 81);
i) Pseudomonas genus (SEQ ID Nos. 21 to 24) and Pseudomonas species (SEQ ID Nos. 82 to 86);
j) Serratia genus (SEQ ID Nos. 25 to 26) and Serratia species (SEQ ID Nos. 87 to 90);
k) Staphylococcus genus (SEQ ID Nos. 27 to 28) and Staphylococcus species (SEQ ID Nos. 91 to 95); and
l) Streptococcus genus (SEQ ID Nos. 29 to 30) and Streptococcus species (SEQ ID Nos. 96 to 104).

51. The method according to claim 47, wherein the identification and detection are done at once for more than one sepsis-causing bacteria species selected from a group consisting of the following members:
a) gram positive bacteria (SEQ ID Nos. 1 to 2) and gram negative bacteria (SEQ ID Nos. 3 to 6);
b) Bacteroides genus (SEQ ID Nos. 7 to 10) and Bacteriodes species (SEQ ID Nos. 31 to 40);
c) Enterococcus genus (SEQ ID Nos. 11 to 12) and Enterococcus species (SEQ ID Nos. 41 to 47);
d) Enterobacter genus (SEQ ID Nos. 17) and Enterobacter species (SEQ ID Nos. 48 to 55);
e) Escherichia coli species (SEQ ID Nos. 56 to 58);
f) Haemophilus genus (SEQ ID Nos. 13) and Haemophilus species (SEQ ID Nos. 59 to 66);
g) Klebsiella genus (SEQ ID Nos. 14 to 17) and Klebsiella species (SEQ ID Nos. 67 to 72);
h) Listeria genus (SEQ ID Nos. 18 to 20) and Listeria species (SEQ ID Nos. 73 to 81);
i) Pseudomonas genus (SEQ ID Nos. 21 to 24) and Pseudomonas species (SEQ ID Nos. 82 to 86);
j) Serratia genus (SEQ ID Nos. 25 to 26) and Serratia species (SEQ ID Nos. 87 to 90);
k) Staphylococcus genus (SEQ ID Nos. 27 to 28) and Staphylococcus species (SEQ ID Nos. 91 to 95); and
l) Streptococcus genus (SEQ ID Nos. 29 to 30) and Streptococcus species (SEQ ID Nos. 96 to 104).

52. The method according to claim 48, wherein the identification and detection are done at once for more than one sepsis-causing bacteria species selected from a group consisting of the following members:
a) gram positive bacteria (SEQ ID Nos. 1 to 2) and gram negative bacteria (SEQ ID Nos. 3 to 6);
b) Bacteroides genus (SEQ ID Nos. 7 to 10) and Bacteriodes species (SEQ ID Nos. 31 to 40);
c) Enterococcus genus (SEQ ID Nos. 11 to 12) and Enterococcus species (SEQ ID Nos. 41 to 47);
d) Enterobacter genus (SEQ ID Nos. 17) and Enterobacter species (SEQ ID Nos. 48 to 55);
e) Escherichia coli species (SEQ ID Nos. 56 to 58);
f) Haemophilus genus (SEQ ID Nos. 13) and Haemophilus species (SEQ ID Nos. 59 to 66);
g) Klebsiella genus (SEQ ID Nos. 14 to 17) and Klebsiella species (SEQ ID Nos. 67 to 72);
h) Listeria genus (SEQ ID Nos. 18 to 20) and Listeria species (SEQ ID Nos. 73 to 81);
i) Pseudomonas genus (SEQ ID Nos. 21 to 24) and Pseudomonas species
j) Serratia genus (SEQ ID Nos. 25 to 26) and Serratia species (SEQ ID Nos. 87 to 90);
k) Staphylococcus genus (SEQ ID Nos. 27 to 28) and Staphylococcus species (SEQ ID Nos. 91 to 95); and
l) Streptococcus genus (SEQ ID Nos. 29 to 30) and Streptococcus species (SEQ ID Nos. 96 to 104).
